(19) United States
(12) Patent Application Publication
(10) Pub. No.: US 2025/0377196 A1
(43) Pub. Date: Dec. 11, 2025

(54) SHAPE AND/OR POSE SENSING USING A HYBRID SENSOR APPROACH

(71) Applicants: FBGS INTERNATIONAL NV, Geel (BE); FBGS TECHNOLOGIES GMBH, Jena (DE); KATHOLIEKE UNIVERSITEIT LEUVEN, Leuven (BE)

(72) Inventors: Omar AL-AHMAD, Heverlee (BE); Jan VAN ROOSBROECK, Hulshout (BE); Johan VLEKKEN, Diepenbeek (BE); Xuan Thao HA, Leuven (BE); Emmanuel VANDER POORTEN, Mechelen (BE); Mouloud OURAK, Leuven (BE); Gianni BORGHESAN, Sint-Pieters-Woluwe (BE)

(21) Appl. No.: 18/878,231

(22) PCT Filed: Jul. 4, 2023
(86) PCT No.: PCT/EP2023/068278

§ 371 Date: Dec. 23, 2024

(30) Foreign Application Priority Data
Jul. 4, 2022 (EP) 22182927.8
Mar. 27, 2023 (EP) 23164501.1

(51) Int. Cl. G01B 11/16; A61B 1/005

(52) U.S. Cl. CPC G01B 11/18; A61B 1/009

(57) ABSTRACT

A method and system are for determining information regarding a shape and/or pose of at least one elongated element. The system includes at least one fiber optic sensor with a multicore optical fiber, at least two electromagnetic sensors, and a controller configured to determine information regarding the shape and/or pose of the at least one elongated element in the reference frame external to the multicore optical fiber.

### FIELD OF THE INVENTION

The present invention relates to the field of shape or pose sensing. More particularly, the present invention relates to methods and systems combining electromagnetic sensing and fiber optic shape sensing for improving accuracy in shape and/or pose sensing for e.g.-but not limited to-medical applications.

### BACKGROUND OF THE INVENTION

Where possible during surgery or medical examination, one attempts to use minimal invasive surgery or examination rather than open surgery. The latter is advantageous, since it reduces surgical risks as well as pain and it speeds up recuperation after the medical intervention. When applying minimal invasive surgery or examination, often use is made of a catheter or endoscope, further referred to as elongated element.

In order to provide information to the surgeon regarding the procedure, the insertion of the elongated element in the body often is done whilst applying medical imaging, in order to assist the surgeon in the procedure, e.g. in deciding where the elongated element is currently positioned and what direction the elongated element needs to go. Different medical imaging techniques are available, but at least some of them suffer from the fact that hazardous radiation of/in the body is required.

For determining a direction to go or a position where the elongated element is positioned, use can also be made of an optical multicore fiber, since the shape of an optical multicore fiber can be determined based on optical signals stemming therefrom. A Fiber Optic (FO) Shape Sensor typically consists of a Multicore Optical Fiber that has multiple Fiber Bragg Gratings (FBG's) inscribed at different points along its length. Via the gratings, each core can measure the local strain. The local strain in general consists of longitudinal and bending strain. The longitudinal strain is identical in all the cores whereas the bending strain is different in each core. By comparing the strain in all cores at a specific position, two parameters, namely curvature (bending radius) and curvature direction (torsion) can be determined at this position. By interpolating the 2 parameters also in between the sensor points, the curvature and curvature direction can be estimated or calculated over the whole sensing length of the fiber. The knowledge of these 2 parameters over the complete sensing length contains essential information to reconstruct the fiber shape. Methods and systems are known, e.g. from U.S. Pat. No. 7,813,599B2, for determining shape using a Fiber Optic shape sensor. The technology allows—due to the small fiber size—for easy integration in slender tools like for example catheters. Furthermore, multiple gratings can be read out simultaneously so that relatively longer sensing lengths can be monitored. Typical lengths range from some tens of centimeters up to 1 m, or longer.

Electromagnetic (EM) position sensors—henceforth referred to as ‘EM-sensors’—positioned in the field of an EM field generator have the ability to measure their absolute position in space (x, y, z coordinates) together with their absolute orientation in space (pitch, roll, yaw). The combination of position and orientation is typically referred to as a “pose”. The field generator serves as the origin of the absolute reference frame. The EM-sensors typically are small in size and consist basically out of a coiled wire placed in a cylindrical body. Their small size as well as their good accuracy (millimeter sized position accuracy and orientation accuracy below 1°) renders them suitable for e.g. real-time tool tracking or navigation in minimal invasive surgery, e.g. in catheters.

Depending on the detected fields and the way these are modulated, the absolute position and orientation can be determined with high accuracy at the specific position where the electromagnetic sensor is placed. Such systems are commercially available.

There are 2 main categories of EM-sensors on the market. EM-sensors allowing to provide information on 5 Degrees Of Freedom (5DOF) and EM-sensors allowing to provide information on 6 Degrees Of Freedom (6DOF). The 5DOF sensors can measure their position (x, y, z coordinates) together with 2 orientation angles (pitch and yaw). The 2 angles determine the orientation of the sensor in space. The 6DOF sensors can in addition also measure the absolute rotation of the sensor around its own longitudinal axis (roll). A 6DOF sensor typically consists of a combination of two 5DOF sensors, whereby information on the additional degree of freedom is actually achieved by combining measurements of two 5DOF sensors. Therefore, a 6DOF sensor is typically larger in size compared to a 5DOF sensor because it contains 2 separate 5DOF sensors in its housing. The different Degrees Of Freedom (DOF) are illustrated in FIG. 1. Three DOF correspond with positional data along the X, Y and Z axis. Three further DOF correspond with rotational data, i.e. rotation about the X-axis (longitudinal direction of the electromagnetic sensor) referred to as roll, rotation about the Y-axis referred to as pitch and rotation about the Z-axis referred to as yaw.

A fiber optic shape sensor does not allow to sense the absolute position and orientation. It is also typically not able to directly detect rotation gradients of the fiber around its own axis, so-called twist. The presence of twist and the difficulty to measure it accurately can result into significant estimation errors of the measured shape.

FIG. 2 provides an illustration helping to understand the fiber twist phenomenon. A regular MCF fiber has straight cores along its length. When subjected to external bending forces or moments, the cores of the MCF would merely curve and bend according to the external load. By measuring the strain in the different cores of the MCF, the curvature and bending direction can be measured in the reference frame of the fiber. On the other hand, when an external rotational force or torsion is applied upon the MCF, the outer cores will twist along their own longitudinal axis (as shown in the bottom illustration of FIG. 2). One thus can make a distinction between two main mechanical deformation cases subjected to the outer cores of the multicore fiber: (1) bending induced and (2) twist induced strain. However, the twist induced strain is in general much smaller than the bending induced strain and in most cases it is too small to be detected. Further, the direction of the twist cannot be measured (clock-wise or anti-clockwise). However, twist will affect the angle of the measurement of the curvature angle and hence results into a wrong calculated shape of the MCF.

A possible methodology to reduce the problem of fiber twist is to use a “helically twisted’ MCF. This is a MCF where the fiber is intentionally twisted during the drawing process to create inherently helically twisted cores within the MCF. This is mainly done to increase the strain sensitivity of the MCF gratings in the outer cores towards induced twist. In addition the direction of twist (clock-wise or anti clockwise) can also be measured. The method has been validated for a fiber with a twist pitch of 3 cm (one full rotation over a length of 3 cm). Although this “twisted” MCF has a higher twist induced sensitivity, the sensitivity is still limited and even higher twist rates are required to make the method really workable. Further decreasing the twist pitch becomes also technically more challenging and eventually it will be fundamentally limited by the length of the Fiber Bragg Gratings.

Another method to mitigate fiber twist effects is to package the fiber within a tube that has a high resistance against torsional forces and in this way prevents the fiber from being twisted. However, this solution cannot fully exclude twist effects.

In WO2020/178336, a technique is described for determining a shape that is less sensitive to twist effects and which makes use of the insertion length. Nevertheless, the technique does not allow for compensating for twist in free space.

In United States patent U.S. Pat. No. 10,772,485B2, methods and systems are described by making use of a hybrid approach by combining an electromagnetic sensor, a marker and a fiber optic shape sensor. For this method to work, it is proposed to have a twist resistant feature configured at the base of the elongated shaft. The effect of such a twist resistant feature may nevertheless be imperfect making it unclear how much twist remains. Furthermore, the method can only work in case the gratings are distributed over the complete length between the base of the elongated shaft until the EM sensor. Finally the EM sensor and marker need also to be correlated in space, which is not straight forward if the distance between both sensors is large.

There is still room for improving, e.g. the accuracy, of systems and methods for determining shape or pose, e.g. in catheter applications.

### SUMMARY OF THE INVENTION

It is an object of embodiments of the present invention to provide good systems and/or methods for providing shape and/or pose information, for example in artery applications such as for example medical applications such as catheter applications.

It is especially advantageous that systems and methods are provided that allow for determining the shape and/or pose of an elongated element, such as for example a catheter, in free space, as can for example be used in ablation medical procedures.

It is an advantage of embodiments of the present invention that data fusion of electromagnetic sensing and optical fiber based sensing is performed for obtaining shape and/or pose information with good accuracy.

It is an advantage of embodiments of the present invention that the limited field of use of EM-sensors can be overcome by combining the EM-sensors with optical fiber shape sensing.

It is an advantage of embodiments of the present invention that accurate shape and/or pose sensing can be obtained by combining point information provided by EM-sensors in an accurate manner with further information from optical fiber shape sensors. In this way point information regarding 3, 4, 5 or 6 DOF can be extended to shape information of an object in between individual measurement points of the electromagnetic sensors.

It is an advantage of embodiments of the present invention that data fusion by combining EM-sensing and Fiber Optic sensing allows to overcome the problems of lack of absolute position and orientation and of fiber rotation or twist encountered by Fiber Optic sensing and the problems of full shape determination encountered by EM-sensing.

It is an advantage of embodiments of the present invention that these allow improvement of accuracy and applicability for shape and pose sensing compared to prior art systems.

It is an advantage of embodiments of the present invention that by data fusion of EM-sensors and fiber optic sensing methods and systems are provided that increase the accuracy of shape and pose sensing.

It is an advantage of embodiments of the present invention that no need is to be made of twisted multi-core fibers, since these are more complex to fabricate.

As illustrated in other examples of the present invention, the integration of extra EMT sensors does not only help localize the reconstructed shape in a fixed coordinate frame but also improves the shape sensing accuracy.

The above objective is accomplished by a method and system according to the present invention.

In one aspect, the present invention relates to a system for determining information regarding a shape and/or pose of at least one elongated element, the system comprising
at least one fiber optic sensor comprising a multicore optical fiber having multiple Fiber Bragg Gratings inscribed along the length of the multicore optical fiber,
at least two electromagnetic sensors, each of the at least two electromagnetic sensors being adapted for providing information regarding their position and/or orientation in a reference frame external to the multicore optical fiber, the at least two electromagnetic sensors being positioned with respect to the at least one fiber optic sensor so as to provide information regarding one or more distinct positions and/or orientations of the fiber along the at least one fiber optic sensor,
a controller configured for determining information regarding the shape and/or pose of the at least one elongated element in the reference frame external to the multicore optical fiber,
the controller being configured for
determining a shape and/or pose construction as function of at least one parameter, based on information from a first and/or second of the at least two electromagnetic sensors and/or based on information from the at least one fiber optic sensor, and
comparing further information of the first and/or second of the at least two electromagnetic sensors and/or of the at least one fiber optic sensor with information of the shape and/or pose construction for deriving therefrom the at least one parameter and for selecting based thereon a preferred shape and/or pose construction.

It is to be noted that whereas in the present invention reference is made to the use of at least one multicore fiber, it is considered that under principle of equivalence, the system alternatively may make use of a bundle of single core fibers, thus considered to be also encompassed by the claimed invention.

Where in embodiments of the present invention reference is made to a shape or pose construction, reference is made to an explicit, an implicit or a parametric representation (e.g. equation) of the shape or pose reconstruction. In some examples reference may be made to a parametric shape or pose reconstruction. Nevertheless, it will be clear that the reconstruction may be equally represented also via an explicit or an implicit representation as well as by a parametric representation.

It is to be noted that where in embodiments of the present invention reference may be made to any type of parameter. As will be further discussed below, the parameter may directly refer to a direct parameter such as for example a twist rate, rotation angle, . . . but may also relate to an indirect characteristic such as a position or other characteristic of a control point of a certain curve such as a Bezier curve, a B-spline curve, a non-uniform rational basis spline (NURBS), a Hermite curve, a general parametric curve, or an arc length between two optical fibers. In this way, the above may refer to an explicit twist compensation approach or an implicit twist compensation approach as illustrated further below.

Where in embodiments of the present invention reference is made to rotation (of a fiber), reference is made to rotation of the point from where the construction of the fiber shape is made. This typically may be the first or last FBG which is taken as the reference frame of the fiber, although embodiments are not limited thereto.

Where reference is made to a controller reference may be made to a system comprising one or more of a processor, a means for data acquisition and a means to perform an algorithm to estimate for example—in real-time or at predetermined or envisioned moments in time—parameters or shapes.

The positions of the electromagnetic sensors thereby typically are in the sensing zone of the fiber.

Where in embodiments of the present invention reference is made to orientation, reference may be made to a combination of yaw and pitch.

The electromagnetic sensors may be positioned dynamically or statically with respect to the at least one fiber optic sensor.

The elongated element may for example be an elongated medical device such as for example a catheter, an endoscope, a needle, a guidewire, etc.

Comparing information may comprise
varying the at least one parameter in the shape and/or pose construction, and
minimizing, as function of the parameter variation,
the difference between a curvature obtained from the shape and/or pose construction and a curvature of the at least one fiber optic sensor, and/or
a quantity depending on a position and/or orientation of one or more of the electromagnetic sensors and the shape and/or pose construction.

In some embodiments, determining a shape and/or pose construction may comprise determining a shape and/or pose construction as function of at least one parameter of the fiber optic sensor that affects the shape and/or pose.

Determining a shape and/or pose construction may be based on information from the first of the at least two electromagnetic sensors and information from the at least one fiber optic sensor.

Comparing further information may comprise comparing information of the second of the at least two electromagnetic sensors and information of the shape and/or pose construction for deriving therefrom the at least one parameter of the fiber optic sensor that affects the shape and/or pose.

Said determining may comprise taking into account a predetermined relation of the at least one parameter of the fiber optic sensor that affects the shape and/or pose along the length of the at least one fiber optic sensor.

The controller may be configured for determining a shape and/or pose construction as function of at least one of a twist parameter and a rotation parameter of the fiber optic sensor.

Alternatively or in addition thereto, the shape and/or pose construction may be a function of one or more of the following parameters: a core-to-center distance, a strain sensitivity, a grating spacing, an intrinsic twist of the fiber, or alike.

Determining may comprise taking into account a predetermined relation of the at least one parameter of the fiber optic sensor that affects the shape and/or pose along the length of the at least one fiber optic sensor.

Alternatively, determining a shape and/or pose construction may take into account a parameter of the fiber optic sensor that affects the shape and/or pose determined based on stochastic information or information obtained based on artificial intelligence.

The predetermined relation may express a constant twist rate along the length and/or a rotation, or the predetermined relation may be a polynomial or other nonlinear relation of the twist rate as function of the distance along the fiber and/or a rotation.

Determining a shape and/or pose construction may comprise building up a shape and/or pose construction starting from the position of the first electromagnetic sensor and taking into account the orientation of the first electromagnetic sensor.

Comparing information may comprise-varying the at least one parameter of the fiber optic sensor in a parameter space, and performing one of
minimizing an Euclidian distance between a position of the second electromagnetic sensor and a corresponding position along the shape and/or pose construction,
minimizing a difference between an orientation of the second sensor and a corresponding orientation along the shape and/or pose construction, or
minimizing a difference between a position and orientation of the second electromagnetic sensor and a corresponding position respectively orientation along the shape and/or pose construction, or
minimizing one or more distances between specific points of the shape and/or pose construction relative to an external body and/or anatomic reference
minimizing misalignment, deformation energy, temporal variation, or any other meaningful characteristic parameter or property linked to the shape and/or pose reconstruction.

In some embodiments the first electromagnetic sensor may be positioned at the start of the zone of the fiber that is to be monitored and the second electromagnetic sensor may be positioned at the end of the zone to be monitored. The start of the zone to be monitored may be the start of the sensing zone of the fiber and the end of the zone to be monitored may be the end of the sensing zone. The end may be the tip of the fiber. Alternatively, one or both of the electromagnetic sensors may be positioned at another position along the sensing zone of the fiber.

Where reference is made to the sensing zone of the fiber, typically reference is made to the zone of the fiber provided with fibre Bragg gratings (FBGs).

In such embodiments, the first electromagnetic sensor may be referred to as the base electromagnetic sensor and the second electromagnetic sensor may be referred to as the distal electromagnetic sensor. It is to be noted that the base electromagnetic sensor could also be positioned at the tip of the fiber. In such cases, rather than referring to the distal electromagnetic sensor, the second sensor may be referred to as the proximal electromagnetic sensor.

The position of at least one of the electromagnetic sensors may change with respect to the fiber optic sensors and the position of the at least one EM sensor may be dynamically updated in the controller. The latter may for example be performed using principles as described in international patent application WO2020/178336.

The twist parameter may be a twist rate which is varied between −2π and 2π radians over the total fiber length and/or the rotation parameter is the amount of rotation which is varied between −π to π. For the specific applications, specific ranges, e.g. different ranges, may be adopted.

The two electromagnetic sensors may be electromagnetic sensors having their longitudinal axis aligned with the longitudinal axis of the fiber optic sensor, at their respective positions.

The electromagnetic sensors may have 5 or 6 DOF but do not need to. For example in one example, the first electromagnetic sensor may have 5 or 6 DOF and the second electromagnetic sensor may have 3 DOF, 4 DOF, 5 DOF or 6 DOF. When the second electromagnetic sensor has at least 5 DOF, the orientation at the corresponding position can also be taken into account.

Where reference is made to an electromagnetic sensor being adapted for providing information regarding at least 5 DOF, reference may be made to an electromagnetic sensor providing information regarding 5 DOF, also referred to as a 5DOF electromagnetic sensor, or an electromagnetic sensor providing information regarding 6 DOF, also referred to as a 6DOF electromagnetic sensor.

It is an advantage of at least some embodiments of the present invention that 5DOF sensors can be used so that the required space can be minimized compared to 6DOF sensors.

The fiber optic sensor is configured to have rotational freedom around its axial direction with respect to the electromagnetic sensors. The fiber optic sensor thus is rotationally decoupled from the electromagnetic sensors. In some embodiments, the rotational degree of freedom of the fiber around its axis with respect to the two electromagnetic sensors may be realised by the shape fiber optic sensor being positioned loosely in a tube or elongated element, and the electromagnetic sensors being coupled to the tube or elongated element so that they do not obstruct internal rotation of the shape fiber optic sensor inside the tube or elongated element. Where in embodiments of the present invention reference is made to the electromagnetic sensors being fixed to the tube or elongated element, such fixation may be at the outside or at the inside of the tube or the elongated element.

In one particular embodiment, the first electromagnetic sensor may have at least 5 DOF and the second electromagnetic sensor may have at least 3 DOF and the fiber optic sensor may be configured to have rotational freedom around its axial direction with respect to both electromagnetic sensors.

The fiber may be mounted loose end in a tube and wherein determining a shape and/or pose construction takes into account that the twist rate at the end of the multicore optical fiber is zero.

Determining a shape and/or pose construction may comprise determining the shape and/or pose construction for one, more or each section between two or more consecutive electromagnetic sensors of the at least two electromagnetic sensors as a parameterized curve. The curve may for example be one or a combination of a Bézier curve, a B-spline, a Hermite curve, a NURBS curve, a parameterized curve or a general implicit curve.

In some embodiments, a construction may be made for sections between each pair of consecutive electromagnetic sensors, but a construction may also be made for a section through multiple EM sensors, e.g. by fitting a single curve, e.g. a single higher order curve.

Determining a shape and/or pose construction may be based at least on information from the position and orientation of the two or more consecutive electromagnetic sensors.

Comparing further information may comprise comparing the curvature along the curve(s) with the curvature as measured with the fiber optic sensor.

Comparing further information may comprise comparing the length of the curve(s) between consecutive electromagnetic sensors with the physical length of the fiber optic sensor between the consecutive electromagnetic sensors.

The parameter may comprise information regarding the control point(s) or other characteristics of the curve(s).

The system may comprise at least 3 electromagnetic sensors and wherein the shape and/or pose construction of the elongated element is built by multiple functions.

The parameterized curve(s) may be 3^{rd} or 4^{th} order or other order Bézier curves.

The first electromagnetic sensor may have at least 5 DOF, and the second electromagnetic sensor may have at least 3 DOF.

The at least two electromagnetic sensors may be electromagnetic sensors having their longitudinal axis aligned with the longitudinal axis of one or more of the fiber optic sensors at their respective positions.

The fiber optic sensor may be rotationally decoupled from the at least two electromagnetic sensors.

Where reference is made to rotationally decoupling, reference is made to the fact that the roll information of the EM sensor is not coupled with the orientation of the fiber.

Rotational decoupling of the fiber optic sensor with respect to the electromagnetic sensors may be established by the shape fiber optic sensor being positioned loosely in a tube and the electromagnetic sensors being coupled to the tube so that they do not obstruct internal rotation of the shape fiber optic sensor inside the tube.

The position of at least one of the electromagnetic sensors can change with respect to the fiber optic sensors and the position of the at least one EM sensor with respect to the fiber may be dynamically updated in the controller.

One electromagnetic sensor may be positioned with respect to one fiber optic sensor and the second electromagnetic sensor may be positioned with respect to another fiber optic sensor. The controller may be adapted for correlating the position of the two electromagnetic sensors with respect to the at least one of the fiber optic sensors. Such system could be for example an embodiment whereby one fiber optic sensor is positioned in a sleeve and a second fiber optic sensor is positioned in a catheter that is passing through said sleeve or it could be a system whereby a optic fiber is introduced in a guidewire and a second optic fiber is introduced in a hollow catheter that makes use of this guidewire to advance carefully through the vasculature, or a setup whereby any kind of longitudinal instrument is used together with a second longitudinal instrument and where by mechanic construction the pair of longitudinal instruments are configured such that they run over a sufficiently long portion of their length along sufficiently parallel paths. Such instruments being for example needles, catheters, guidewires, flexible endoscopes, dilators, sheaths or other longitudinal flexible medical devices.

The controller may be adapted for correlating the position of the two electromagnetic sensors with respect to the at least one of the fiber optic sensors based on shape and/or pose information obtained from a coinciding portion of both fiber optic sensors or from historic data, or from knowledge of the relative motion of the fiber optic sensors and/or electromagnetic sensors e.g. by some external measurement system.

At least one of the at least two electromagnetic sensors may be a virtual electromagnetic sensor representing a historic or memorized position and/or pose of a non-virtual electromagnetic sensor. The virtual electromagnetic sensor may be used to provide information of a distinct position and/or orientation of the fiber along the at least one fiber optic sensor.

For determining for example a position of the virtual electromagnetic sensor with respect to the fiber, one can use methods as known from prior art. For determining the position of the virtual electromagnetic sensor with respect to the fiber, for example the travelled path of a non-virtual electromagnetic sensor may be used or other methods as known from the prior art may be used.

Where reference is made to at least two electromagnetic sensors, reference may thus in some embodiments be made to two real and physical electromagnetic sensors but in alternative embodiments may refer to one real electromagnetic sensor and a virtual electromagnetic sensor, or two virtual electromagnetic sensors, whereby data of the virtual electromagnetic sensor actually refers to memorized or historical data previously recorded with a real electromagnetic sensor, e.g. a real one electromagnetic sensor referred to above. If the position of the elongated body at the level of the virtual EM sensor does not alter over time or alters in a known or measurable fashion this virtual sensor could thus replace a real sensor. Combinations of multiple virtual and real EM sensors can be employed i.e. not necessarily restricted to 2.

In one aspect, the present invention relates to a method for determining information regarding a shape and/or pose of at least one an elongated element, the method comprising
determining a shape and/or pose construction as function of at least one parameter, based on information from a first and/or second of at least two electromagnetic sensors and/or based on information from at least one fiber optic sensor, and
comparing further information of the first and/or second of the electromagnetic sensors and/or of the at least one fiber optic sensor with information of the shape and/or pose construction for deriving therefrom the at least one parameter and for selecting based thereon a preferred shape and/or pose construction.

Further optional method steps may correspond with the functionality of features of the system as described above.

The present invention in one aspect also relates to a system for determining information regarding a shape and/or pose of an elongated element, the system comprising
at least one fiber optic sensor comprising a multicore optical fiber having multiple Fiber Bragg Gratings inscribed along the length of the multicore optical fiber,
at least two electromagnetic sensors, each of the two electromagnetic sensors being adapted for providing information regarding their position and/or orientation in a reference frame external to the multicore optical fiber, the two electromagnetic sensors being positioned with respect to the at least one fiber optic sensor so as to provide information regarding two distinct positions and/or orientations of the fiber along the at least one fiber optic sensor, and the fiber optic sensor being rotationally decoupled from the at least two electromagnetic sensors,
a controller configured for determining information regarding the shape and/or pose of the elongated element in the reference frame external to the multicore optical fiber, the controller being configured for
determining a parameterized shape and/or pose construction as function of at least one parameter of the fiber optic sensor that affects the shape and/or pose, based on information from a first of the at least two electromagnetic sensors and information from the at least one fiber optic sensor, and
comparing information of the second of the at least two electromagnetic sensors and information of the parameterized shape and/or pose construction for deriving therefrom the at least one parameter of the fiber optic sensor that affects the shape and/or pose.

Where reference is made to rotationally decoupling, reference is made to the fact that the roll information of the EM sensor is not coupled with the orientation of the fiber.

Where reference is made to a controller reference may be made to a system comprising one or more of a processor, a means for data acquisition and a means to perform an algorithm to estimate for example—in real-time or at predetermined or envisioned moments in time—parameters or shapes

The positions of the electromagnetic sensors thereby typically are in the sensing zone of the fiber.

Where in embodiments of the present invention reference is made to orientation, reference may be made to a combination of yaw and pitch.

The electromagnetic sensors may be positioned dynamically or statically with respect to the at least one fiber optic sensor.

The elongated element may for example be an elongated medical device such as for example a catheter, an endoscope, etc.

The parameterized shape and/or pose construction may be a function of a twist parameter, a function of a rotation parameter or a function of a twist parameter and a rotation parameter.

Alternatively or in addition thereto, the parameterized shape and/or pose construction may be a function of one or more of the following parameters: a core-to-center distance, a strain sensitivity, a grating spacing, an intrinsic twist of the fiber, or alike.

Determining may comprise taking into account a predetermined relation of the at least one parameter of the fiber optic sensor that affects the shape and/or pose along the length of the at least one fiber optic sensor.

Alternatively, determining a parameterized shape and/or pose construction may take into account a parameter of the fiber optic sensor that affects the shape and/or pose determined based on stochastic information or information obtained based on artificial intelligence.

The controller may be configured for determining a parameterized shape and/or pose construction as function of at least one of a twist parameter and a rotation parameter of the fiber optic sensor.

In preferred embodiments, the EM sensor may be oriented parallel with the axis around which role is defined, for example in FIG. 1 this would be parallel to the x-axis.

The predetermined relation may express a constant twist rate along the length and/or rotation or is a polynomial or any other convenient nonlinear relation of the twist rate as function of the distance and/or rotation.

Comparing information may comprise varying the at least one parameter of the fiber optic sensor in a parameter space, and performing one of
minimizing an Euclidian distance between a position of the second electromagnetic sensor and a corresponding position along the parameterized shape and/or pose construction, optimizing a difference between an orientation of the second electromagnetic sensor and a corresponding orientation along the parameterized shape and/or pose construction, and
optimizing a difference between a position and orientation of the second electromagnetic sensor and a corresponding position respectively orientation along the parameterized shape and/or pose construction.

Such an orientation may for example be derived from a tangential direction of the parameterized shape and/or pose construction at the position of the second electromagnetic sensor.

Optimizing also may comprise taking into account additional information, such as for example imaging information, although embodiments are not limited thereto.

In general, twist can vary randomly over the fiber length, depending on how the twist is applied and what the fiber friction is. However, in some cases, the twist can be assumed to be constant over the fiber length. In this case, it can be indicated with a constant parameter that will be referred to as the ‘twist rate’.

The twist rate may be varied between −2π and 2π radians over the total sensing zone of the fiber and/or the rotation parameter which is the amount of rotation of the fiber at for example the start of the sensing zone, may be varied between −π to π. For the convenience of specific applications different ranges may be adopted.

The two electromagnetic sensors may be electromagnetic sensors having their longitudinal axis aligned with the longitudinal axis of the fiber optic sensor, at their respective positions.

The electromagnetic sensors may have 5 or 6 DOF but do not need to. For example in one example, the first electromagnetic sensor may have 5 or 6 DOF and the second electromagnetic sensor may have 3 DOF, 4 DOF, 5 DOF or 6 DOF. When the second electromagnetic sensor has at least 5 DOF, the orientation at the corresponding position can also be taken into account.

Where reference is made to an electromagnetic sensor being adapted for providing information regarding at least 5 DOF, reference may be made to an electromagnetic sensor providing information regarding 5 DOF, also referred to as a 5DOF electromagnetic sensor, or an electromagnetic sensor providing information regarding 6 DOF, also referred to as a 6DOF electromagnetic sensor.

It is an advantage of at least some embodiments of the present invention that 5DOF sensors can be used so that the required space can be minimized compared to 6DOF sensors.

The fiber optic sensor is configured to have rotational freedom around its axial direction with respect to the electromagnetic sensors. The fiber optic sensor thus is rotationally decoupled from the electromagnetic sensors. In some embodiments, the rotational degree of freedom of the fiber around its axis with respect to the two electromagnetic sensors may be realised by the shape fiber optic sensor being positioned loosely in a tube or elongated element, and the electromagnetic sensors being coupled to the tube or elongated element so that they do not obstruct internal rotation of the shape fiber optic sensor inside the tube or elongated element. Where in embodiments of the present invention reference is made to the electromagnetic sensors being fixed to the tube or elongated element, such fixation may be at the outside or at the inside of the tube or the elongated element.

In one particular embodiment, the first electromagnetic sensor may have at least 5 DOF and the second electromagnetic sensor may have at least 3 DOF and the fiber optic sensor may be configured to have rotational freedom around its axial direction with respect to both electromagnetic sensors.

In some embodiments the first electromagnetic sensor may be positioned at the start of the zone of the fiber that is to be monitored and the second electromagnetic sensor may be positioned at the end of the zone to be monitored. The start of the zone to be monitored may be the start of the sensing zone of the fiber and the end of the zone to be monitored may be the end of the sensing zone. The end may be the tip of the fiber. Alternatively, one or both of the electromagnetic sensors may be positioned at another position along the sensing zone of the fiber.

Where reference is made to the sensing zone of the fiber, typically reference is made to the zone of the fiber provided with FBGs.

In such embodiments, the first electromagnetic sensor may be referred to as the base electromagnetic sensor and the second electromagnetic sensor may be referred to as the distal electromagnetic sensor. It is to be noted that the base electromagnetic sensor could also be positioned at the tip of the fiber. In such cases, rather than referring to the distal electromagnetic sensor, the second sensor may be referred to as the proximal electromagnetic sensor.

The position of at least one of the electromagnetic sensors may change with respect to the fiber optic sensors and the position of the at least one EM sensor may be dynamically updated in the controller. The latter may for example be performed using principles as described in international patent application WO2020/178336.

The fiber may be mounted loose end in a tube and determining a parameterized shape and/or pose construction may take into account that the twist rate at the end of the multicore optical fiber is zero.

One electromagnetic sensor may be positioned with respect to one fiber optic sensor and the second electromagnetic sensor may be positioned with respect to another fiber optic sensor. The controller may be adapted for correlating the position of the two electromagnetic sensors with respect to at least one fiber optic sensor, by looking at overlapping segments of both fiber optic sensors.

In one aspect, the present invention also relates to a method for determining information regarding a shape and/or pose of an elongated element, the method comprising determining information regarding the shape and/or pose of the elongated element in the reference frame external to the multicore optical fiber, said determining comprising
determining a parameterized shape and/or pose construction as function of at least one parameter of the fiber optic sensor that affects the shape and/or pose, based on information from a first of the at least two electromagnetic sensors and information from at least one fiber optic sensor, the at least one fiber optic sensor comprising a multicore optical fiber having multiple Fiber Bragg Gratings inscribed along the length of the multicore optical fiber and each of the two electromagnetic sensors being adapted for providing information regarding their position and/or orientation in a reference frame external to the multicore optical fiber, the two electromagnetic sensors being positioned with respect to the at least one fiber optic sensor so as to provide information regarding two distinct positions and/or orientations of the fiber along the at least one fiber optic sensor, and the fiber optic sensor being rotationally decoupled from the at least two electromagnetic sensors, and
comparing information of the second of the at least two electromagnetic sensors and information of the parameterized shape and/or pose construction for deriving therefrom the at least one parameter of the fiber optic sensor that affects the shape and/or pose.

In one aspect, the present invention relates to a system comprising a shape sensing fiber and at least one electromagnetic-sensor, wherein the at least one electromagnetic sensor is positioned concentrically with the shape sensing fiber. The at least one electromagnetic sensor may surround the shape sensing fiber. The at least one electromagnetic sensor may be a hollow electromagnetic sensor.

Further optional method steps may correspond with the functionality of features or elements of embodiments of the system as described in the first aspect.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1 shows the 6 possible DOF in a point of a longitudinal element, as used in embodiments according to the present invention.

FIG. 2 illustrates the effect of twist in a multicore fiber, as can be taken into account when determining shape and/or pose of an elongated element in embodiments according to the present invention.

FIG. 3 illustrates an exemplary method according to an embodiment of the present invention.

FIG. 4 illustrates a view of the graphical user interface of software for determining a shape and/or pose of an elongated element according to an embodiment of the present invention.

FIG. 5 illustrates the experimental setup to investigate EM data fusion for dynamic twist compensation: (A) catheter placed in a straight configuration between a pair of EM sensors, and (B) catheter placed in a curved and planar configuration between a pair of EM sensors, illustrating features of the first additional example.

FIG. 6 shows results of the experiments investigating EM data fusion for dynamic twist compensation: (A) distance errors between EM sensor position and reconstructed shape tip for planar curved catheter motion, (B) example result illustrating the degree in which the reconstructed shape may differ with and without twist compensation, and (C) distance errors between EM sensor position and the reconstructed shape tip for three separate approaches incorporating a different combination of twist compensation parameters. Twist offset and twist gradient magnitudes across the time steps are correspondingly illustrated for the additional first example.

FIG. 7 illustrates the relation between EMT coordinate frame {em} and the FBG local coordinate frame {f} that is coincidentally fixed with the 1st set of gratings is shown in this figure, as used in the third additional example. A side and a cross section view of the multi-core FBG fiber can be seen in a) and b), respectively. The angle of the bending plane and the angle of the 2nd core with respect to the x-axis are denoted as θb and θ2 in b), respectively.

FIG. 8 shows an example of fitting a catheter with 3 integrated EMT sensors by 3^{rd} order Bézier curves, as used in the third particular example. Here, two Bézier curves are utilized to approximate the catheter shape. The first Bézier curve starts at

$${}^{em} {{P}_{{e}_{1}}}^{}$$

and ends at

$$\text{?} .$$

$$\text{?} \text{indicates text missing or illegible when filed}$$

The second Bézier curve starts at

$${}^{em} {P}_{{e}_{2}}$$

and ends at

$$\text{?} .$$

$$\text{?} \text{indicates text missing or illegible when filed}$$

The control points of the first and second Bézier curve are colored in black and gray, respectively.

FIG. 9 illustrates in a) the dimension of the sensorized dilator and how the multi-core FBG fiber is integrated. b) The experimental setup for 2D experiment includes the sensorized dilator, a monocular camera to capture the 2D shape of the dilator, a fan-out (to guide light from a four-core fiber into four separate channels) and an interrogator.

FIG. 10 illustrates an image captured by the camera is visualized in a); b) color segmentation is applied on the input image to segment the contour of the optical markers; c) the centroids of the contours (red markers) of the optical markers are defined as the optical marker positions in the image coordinate frame; d) the set of positions of all optical markers are approximated by a B-spline curve (blue). This B-spline curve is used as the ground truth shape. The tangent vectors (cyan) are calculated by taking the first derivative of the approximated B-spline curve.

FIG. 11 shows a 3D experiment setup containing the EMT field generation, fusionTrack 500 system and a multi-core FBG fiber stylus. three EMT sensors and 6 spherical fiducials are attached along the length of the stylus. The registration fiducial is used to register between the EMT coordinate frame and the fusion Track coordinate frame.

FIG. 12 shows the reconstructed shapes in the image coordinate frame of the 2D experiments using 4 pose sensors can be seen in (a)-(f). The shapes reconstructed by the traditional method (EMT+FBG) is shown in green while the shape reconstructed by the proposed method using 3^{rd} and 4^{th} order Bézier curves (EMT+FBG) are shown in orange and cyan, respectively. The shapes estimated by the EMT-based method are shown in magenta. The blue curves show the ground truth shape. The red circles are the tracked optical markers. Figure (g)-(h) show the error (in mm) of the traditional method (EMT+FBG), the proposed method (EMT+FBG) and the method in which only EMT sensors are used in two cases—using 3 pose sensors (1^{st} case) and using 4 pose sensors (2^{nd} case).

FIG. 13 shows the reconstructed shapes together with the fusionTrack fiducials in the EMT coordinate frame of the 3D experiment. The shapes reconstructed by the traditional method (EMT+FBG) is shown in blue while the shape reconstructed by the proposed method using 4th order Bézier curves is shown in orange (EMT+FBG). The yellow curves show the shape estimation results by using only EMT sensors. The fusionTrack fiducials (green) are transformed to the EMT coordinate frame using the transformation matrix obtained from the preregistration step.

FIG. 14 illustrates a cross section view of the multi-core FBG fiber and the guidance sheath can be seen in (a) and (b), respectively, according to an embodiment of the second particular example. r is the distance between the central and the surrounding core. The angle of the bending plane and the angle of the 2^{nd} core with respect to the x-axis are denoted as θ_{b} and θ₂, respectively. The distance between the center of the guidance sheath and the off-centered channel central axis is denoted as d_{f}. The angle between the x-axis of the fiber in the central channel and the center of the off-centered channel O_{f} is θ_{f}. The fiber sensitive length shown in (c) starts at the first set of grating and ends at the last set of grating.

FIG. 15 illustrates the reconstructed shape of the catheter is localized in the sheath base frame{sh}. The overlapping length of the guidance sheath starts at sᵢ=sᵢₛₜ and ends at sᵢ=sᵢₑₙ while that of the catheter starts at sⱼ=sⱼₛₜ and ends at sⱼ=sⱼₑₙ. The sensitive length of the two fibers is shown in the dashed gray lines.

FIG. 16 shows the proposed fusion framework, wherein the catheter shape is approximated by a Bezier curve that matches best with the shapes estimated by the different sensors. The black lines show the shapes estimated by the traditional FBG-based shape sensing method while the gray lines show the shapes estimated by an EMT and FBG-based shape sensing method.

FIG. 17 illustrates an algorithm as used in the particular second example.

FIG. 18 shows the experiment setup in (a) including the built coaxial catheter system, EMT tracking system, and FBG interrogator. The 3D design of the coaxial catheter system is described in (b). The two EMT sensors are attached to the catheter and the guidance sheath by two 3D printed fixtures. The multi-core fibers are inserted into the central channel of the catheter and the guidance sheath.

FIG. 19 shows the 3D estimated shapes of the guidance sheath and the catheter using the proposed fusion approach and the traditional approach in the EMT coordinate frame projected to the images from the left camera. The ground truth shape of the guidance sheath is shown by the blue circles. The guidance sheath shapes estimated by the proposed fusion approach are plotted in green. The red and black line show the estimated catheter shape using our proposed fusion approach and the traditional approach, respectively. The experimental results of the first and second experiments can be seen in the images on the first row (a)-(f) and second row (g)-(1), respectively.

FIG. 20 shows the quantitative shape tracking results of the two experiments using the traditional approach and our proposed fusion approach. The guidance sheath tracking error (referenced to left y-axis) are calculated in mm while that of the catheter (referenced to the right y-axis) are calculated in pixels.

FIG. 21 shows an example of an implementation of a sensing system according to an embodiment of the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

While the invention is illustrated and described in detail in the drawings and accompanying description, such illustration and description are to be considered illustrative or exemplary and not restrictive. The invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

The mere fact that certain measures are recited in mutually different dependent claims does not indicate that a combination of these measures cannot be used to advantage.

Where in embodiments of the present invention reference is made to a shape and/or pose of an elongated element or a fiber sensor, reference is made to a shape, orientation and/or position of the elongated element or fiber sensor, with respect to a reference system external to the elongated element.

Where in embodiments of the present invention reference is made to parallel orientation, reference is made to an alignment being substantially parallel. Small errors could occur in the alignment due to inaccuracies or inherent alignment procedure characteristics.

Where in embodiments of the present invention reference is made to orientation, reference is made to the pitch and yaw of the orientation.

The following description details certain embodiments of the invention. It will be appreciated, however, that no matter how detailed it appears in text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

In a first aspect, the present invention relates to a system for determining information regarding a shape and/or pose of at least one elongated element. Such an elongated element may for example be a medical elongated element such as for example a catheter, an endoscope or alike, although embodiments of the present invention are not limited thereto. It is an advantage of embodiments of the present invention that they allow for determining information regarding the shape and/or pose of the elongated element with respect to an external reference, i.e. for example with respect to an electromagnetic field generator, e.g. positioned with respect to the body of a patient. According to embodiments, the system comprises at least one fiber optic sensor comprising a multicore optical fiber, such as for example a multicore optical fiber having multiple Fiber Bragg Gratings inscribed along the length of the multicore optical fiber. Alternatively the multicore fiber may be replaced by a bundle of single core fibers, which also is considered to be encompassed in embodiments of the present invention. In one preferred embodiment of the invention, one of the multi-core fibers is a virtual multi-core fiber that is formed by 2, 3 or more single-core optical fibers that are positioned at a fixed geometric relation with respect to each other such that the shape of the virtual multi-core fiber is found from computing the distributed strains in the respective single-core optical fibers and using knowledge of the fixed geometric relation. In one preferred embodiment the single-core optical fibers are e.g. embedded at locations, e.g. separated over 120 degrees, along a guidance sheath. In this embodiment a real multi-core optical fiber could be e.g. embedded in a catheter that passes through the guidance sheath. In other preferred embodiments other combinations of single-core fibers or single-core and multicore optical fibers can be used advantageously.

The different FBGs can be measured using for example (but not limited to) Time Domain Mulitplexing (TDM), Wavelength Division Multiplexing (WDM), Code Division Multiplexing (CDM) or OFDR (Optical Frequency Domain Reflectometry) techniques. In addition, a similar approach can also be realized using Rayleigh backscattering techniques without the usage of FBGs. The latter is also envisaged within the scope of the present invention.

The system also comprises at least two electromagnetic sensors, each of the at least two electromagnetic sensors being adapted for providing information regarding their position and/or orientation in a reference frame external to the multicore optical fiber. The at least two electromagnetic sensors being positioned with respect to the at least one fiber optic sensor so as to provide information regarding one or more distinct positions and/or orientations of the fiber along the at least one fiber optic sensor.

The system also comprises a controller configured for determining information regarding the shape and/or pose of the at least one elongated element in the reference frame external to the multicore optical fiber. The controller is being configured for determining a shape and/or pose construction as function of at least one parameter, based on information from a first and/or second of the at least two electromagnetic sensors and/or based on information from the at least one fiber optic sensor, and comparing further information of the first and/or second of the at least two electromagnetic sensors and/or of the at least one fiber optic sensor with information of the parameterized shape and/or pose construction for deriving therefrom the at least one parameter and for selecting based thereon a preferred shape and/or pose construction.

According to some embodiment the fiber optic sensor may be referenced to the same external reference frame as the electromagnetic sensors. Since the fiber optic sensor is positioned along the elongated element, the shape and/or pose construction of the fiber optic sensor corresponds with a shape and/or pose construction of the elongated element. According to some embodiments, such information may be or comprise one or more parameters of the fiber optic sensor, such as for example a rotation, a twist parameter like a twist rate, a core-to-center distance, a strain sensitivity, a grating spacing, an intrinsic twist of the fiber, or alike. The information may also be the shape and/or pose of the elongated element. According to embodiments, the controller is being configured for determining a parameterized shape and/or pose construction as function of at least one parameter of the fiber optic sensor that affects the shape and/or pose based on information from a first of the at least two electromagnetic sensors and information from the at least one fiber optic sensor, and for comparing information of the second of the at least two electromagnetic sensors and information of the parameterized shape and/or pose construction for deriving therefrom the at least one parameter of the fiber optic sensor that affects the shape and/or pose.

Further features and advantages may correspond with one, more or all of the features and/or advantages of examples and/or embodiments described in the present application. Further features and advantages may in one set of embodiments correspond with one, more or all features and advantages of particular example 1.

In other embodiments, determining a parameterized shape and/or pose construction may comprise determining the parameterized shape and/or pose construction for one, more or each section between two or more consecutive electromagnetic sensors of the at least two electromagnetic sensors as a parameterized curve. The curve may fore example be one or a combination of a Bézier curve, a B-spline, a Hermite curve, a NURBS curve, a parameterized curve or an implicit curve. In some embodiments, a parameterized construction may be made for sections between each pair of consecutive electromagnetic sensors, but a parameterized construction may also be made for a section through multiple EM sensors, e.g. by fitting a single curve, e.g. a single higher order curve.

Determining a parameterized shape and/or pose construction may be based at least on information from the position and orientation of the two or more consecutive electromagnetic sensors. Comparing further information may comprise comparing the curvature along the parameterized curve(s) with the curvature as measured with the fiber optic sensor.

Comparing further information may comprise comparing the length of the parameterized curve(s) between consecutive electromagnetic sensors with the physical length of the fiber optic sensor between the consecutive electromagnetic sensors.

The parameter may comprise information regarding the control point(s) or other characteristics of the curve(s).

The controller may be implemented in software, in hardware or in a hybrid version thereof. It may comprise a processor for performing the different steps. It may operate based on a predetermined algorithm, stochastic models and/or artificial intelligence components.

The two electromagnetic sensors may be electromagnetic sensors having their longitudinal axis aligned with the longitudinal axis of the fiber optic sensor, at their respective positions. The first electromagnetic sensor, which also may be referred to as the base electromagnetic sensor, typically is the electromagnetic sensor from where the shape and/or pose construction of the elongated element is being started. Typically, in embodiments according to the present invention, the position of the first electromagnetic sensor is used as starting position of the shape and/or pose construction and the orientation of the first electromagnetic sensor-assuming that the orientation of the first electromagnetic sensor with respect to the fiber optic sensor is known-is used to define an orientation of the shape and/or pose construction in the starting point. The first electromagnetic sensors therefore advantageously may have 5 or 6 DOF whereas the second electromagnetic sensor may have 3, 4, 5 or 6 DOF. Whereas a 5 DOF electromagnetic sensor provides, aside from information regarding the position, two orientation parameters (pitch and yaw), a 6 DOF electromagnetic sensor provides, aside from information regarding the position, three orientation parameters (pitch, yaw and roll). However, the measurement of the roll is not used as the fiber is rotationally decoupled form the EM-sensor.

The electromagnetic sensors may be positioned dynamically or statically with respect to the at least one fiber optic sensor.

In a first example, embodiments may relate to a predetermined relation of the at least one parameter of the fiber optic sensor that affects the shape and/or pose along the length of the at least one fiber optic sensor is taken into account. Such a predetermined relation may for example be a constant parameter value along the length of the at least one fiber optic sensor or may for example be a polynomial relation, or for example be another, suitable, nonlinear relationship. Alternatively, the information regarding the parameter may be stochastic information or information obtained based on artificial intelligence, such as for example based on a neural network.

In some embodiments, comparing information may comprise minimizing a distance, e.g. an Euclidian distance, between a position of the second electromagnetic sensor and a corresponding position along the parameterized shape and/or pose construction, minimizing a difference between an orientation of the second electromagnetic sensor and a corresponding orientation along the parameterized shape and/or pose construction, or minimizing or optimizing a combination of both the distance and orientation between the second electromagnetic sensor and a corresponding position and orientation along the parameterized shape and/or pose construction. Where reference is made to determining orientation, the latter may for example be derived from a tangential direction of the parameterized shape and/or pose construction at the position of the second electromagnetic sensor. Comparing information may for example be performed by allowing the one or more parameter to vary within a predetermined parameter space and by minimizing e.g. the distance, an orientation or a combination thereof.

In one embodiment, the first electromagnetic sensor may have at least 5 DOF and the second electromagnetic sensor may have at least 3 DOF. One example of such an embodiment is a system wherein a single multicore fiber is used with two electromagnetic sensors, whereby both electromagnetic sensors are rotationally decoupled from the fiber. In this example, two EM-sensors are thus combined with 1 fiber optic sensor. The electromagnetic sensors are fixed to the active shape sensing section, but the fiber can rotate freely with respect to both EM sensors. In this example, the two electromagnetic sensors are positioned along the longitudinal axis of the fiber optic sensor. The longitudinal axis of the EM sensors are aligned with the longitudinal axis of the fiber. One EM-sensor (the proximal EM-sensor) is placed at the base of the fiber (at the start of the sensing zone). This EM-sensor will be the base position from where the shape is constructed. The other EM-sensor (the distal EM-sensor) is placed near the tip of the fiber (at the end of the sensing zone). The rotational freedom of the fiber can for example be realized when the fiber optic sensor is loosely placed in a probe i.e. a shielding tube like for example a nitinol tube of 600 μm diameter, and the EM sensors are fixed inside or outside this nitinol tube in such a way that they don't significantly obstruct the movement of the fiber inside the tube. In this embodiment, advantageously 5 DOF sensors are used thereby minimizing the space used. The fiber can make pure rotations or rotational gradients relative to the EM-sensors. In general, twist can vary randomly over the fiber length, depending on how the twist is applied and what the fiber friction is. Since in some cases the twist can be assumed to be constant over the fiber length, the parameter used in the parameterized shape and/or pose construction is a constant parameter that can be referred to as the ‘twist rate’. The pure rotation and twist rate thus can be considered as fit parameters that can be fitted by means of a minimalization procedure. In the present embodiment, the minimalization procedure comprises the construction of the shape from base/proximal EM-sensor. It fixes the starting point and the 3D orientation of the shape in space. The procedure further comprises using the pure rotation and twist rate as 2 fit parameters that will be varied to within their boundaries. For the present example, rotation is limited from −π to +π and twist rate is varied between −2π and 2π radians over the total sensing length. The combination of rotation and twist rate that minimizes the Euclidian distance in 3D space between the distal EM-sensor and the corresponding fiber shape position could then lead to one embodiment offering a certain optimal fit of the parameters, which allow to determine an optimal shape reconstruction relative to the formulated optimization problem. Other methods to define an optimal shape reconstruction can be defined and implemented along similar lines.

In some embodiments, the position of at least one of the electromagnetic sensors may change with respect to the fiber optic sensors and the position of the at least one EM sensor may be dynamically updated in the controller. The position of the EM sensors with respect to the fiber may be determined using for example techniques as described in International patent application WO2020/178336.

By way of illustration, embodiments of the present invention not being limited thereto, further optional features and advantages may become apparent from the further description and examples illustrated below.

The system according to embodiments of the present aspect, may comprise or use elements for reading out the electromagnetic sensors. This may for example be performed using an electromagnetic field generator and corresponding sensor. The corresponding technology is as such known in the field and consequently, no further elaboration is made here.

The system according to embodiments of the present aspect, may comprise or use elements for reading out a multicore optic fiber sensor. A system for sensing shape may use one or more interrogators, fanout boxes for splitting the different cores of the multicore fiber used and corresponding shape sensing software. The shape sensing software may use wavelength information together with sensor specific parameters (like e.g. the spatial separation of the DTG's, the core to center distance, . . . ) to calculate curvature, curvature angles and eventually also 3D shape. The system may furthermore comprise transport cable for bridging a distance between the sensing part of the multicore fiber and the fanout box(es) and suitable connectors, Since the corresponding technology for sensing shape with an optic fiber sensor is as such known in the field, no further elaboration is made here.

According to embodiments, the system may furthermore be equipped with components or be programmed for performing one, a combination or all of the process steps as described in embodiments of the corresponding method described in the second aspect.

In one exemplary embodiment, the present invention also relates to a system wherein one electromagnetic sensor is positioned with respect to a first fiber optic sensor and the second electromagnetic sensor is positioned with respect to a second fiber optic sensor. The controller is adapted for correlating the position of the two electromagnetic sensors with respect to the at least one of the fiber optic sensors based on shape and/or pose information obtained from a coinciding portion of both fiber optic sensors. The example overcomes the problem that it is not always possible to have 2 electromagnetic sensors along the fiber optic sensor in one device. In this example, there are 2 sets of 1 EM-sensor and 1 fiber optic sensor, and the paths of both fiber optic sensors are at least partially overlapping. For example, the first EM-sensor and fiber optic sensor are placed in a delivery catheter and the second EM-sensor and fiber optic sensor are placed in a sheath catheter that is used to steer the delivery catheter to the right position in the intracardiac region. Since the delivery catheter is progressed in the sheath catheter, both fiber optic sensors can be assumed to partially overlap. The position of overlap can be determined by making a comparison in the curvature profile as measured by both fibers. Since both fibers partially overlap, part of their measured curvature profile will overlap. This can be done very accurately because curvature is not depending on the fiber rotation nor on twist. A minimalization or correlation procedure can be applied to find the best overlap position, leading to knowledge of which grating from the first assembly is closest to the EM-sensor from the second assembly and/or vice versa.

In one aspect, the present invention also relates to a method for determining information regarding a shape and/or pose of an elongated element. The method may for example be performed with a system as described in the first aspect, although embodiments are not limited thereto.

The method for determining information regarding a shape and/or pose of at least one elongated element typically may comprise
determining a shape and/or pose construction as function of at least one parameter, based on information from a first and/or second of at least two electromagnetic sensors and/or based on information from at least one fiber optic sensor, and
comparing further information of the first and/or second of the electromagnetic sensors and/or of the at least one fiber optic sensor with information of the shape and/or pose construction for deriving therefrom the at least one parameter and for selecting based thereon a preferred shape and/or pose construction.

According to some embodiments, the method may for example comprise determining information regarding the shape and/or pose of an optical fiber sensor inserted in or fixed to the elongated element, in the reference frame external to the multicore optical fiber. Such determining comprises determining a parameterized shape and/or pose construction as function of at least one parameter of the fiber optic sensor that affects the shape and/or pose, based on information from a first of the at least two electromagnetic sensors and information from at least one fiber optic sensor, the at least one fiber optic sensor comprising a multicore optical fiber having multiple Fiber Bragg Gratings inscribed along the length of the multicore optical fiber and each of the two electromagnetic sensors being adapted for providing information regarding their position and/or orientation in a reference frame external to the multicore optical fiber, the two electromagnetic sensors being positioned with respect to the at least one fiber optic sensor so as to provide information regarding two distinct positions and/or orientations of the fiber along the at least one fiber optic sensor, and the fiber optic sensor being rotationally decoupled from the at least two electromagnetic sensors. Such determining also comprises comparing information of the second of the at least two electromagnetic sensors and information of the parameterized shape and/or pose construction for deriving therefrom the at least one parameter of the fiber optic sensor that affects the shape and/or pose.

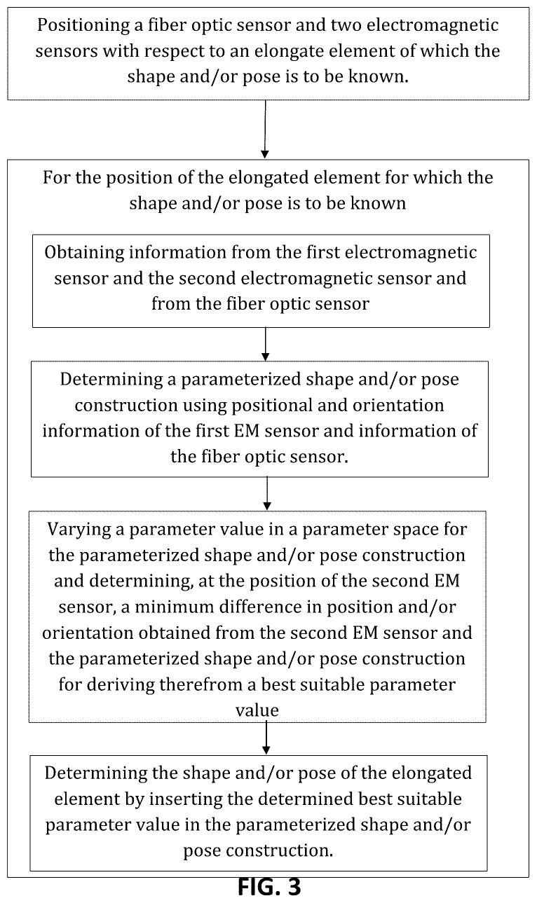

An example of a method according to an embodiment of the present invention is described with reference to FIG. 3.

The exemplary method comprises positioning a fiber optic sensor and two electromagnetic sensors with respect to an elongate element of which the shape and/or pose is to be known. The latter can for example be performed by inserting an optic fiber sensor in the elongated element or fixing the optic fiber sensor to the elongated element. The two electromagnetic sensors typically are positioned along the optic fiber sensor at distinct positions such that information is obtained from different positions. Their position typically is along the optic fiber sensor but may be static or dynamic with respect to the optic fiber sensor.

The elongated element may already be in a position for which one wishes to know the shape and/or pose or may be brought in such a position. For such a position, the exemplary method further comprises the following steps.

The method comprises obtaining information from the first electromagnetic sensor and from the second electromagnetic sensor as well as obtaining information from the fiber optic sensor.

The method also comprises determining a parameterized shape and/or pose construction using positional and orientation information of the first electromagnetic sensor and information of the fiber optic sensor. The positional information of the first electromagnetic sensor provides a fixed position for the shape and/or pose construction. The shape and/or pose construction is constructed starting from this fixed position. The orientation information of the first electromagnetic sensor provides orientational information for the shape and/or pose construction in that fixed position. Typically the first electromagnetic sensor will have an at least partly fixed orientation with respect to the fiber optic sensor and this orientation information will be used for determining an orientation of the shape construction in that fixed position. For example, the orientation information may provide an orientation for the tangent to the shape and/or pose construction in the fixed position. Determining a parameterized shape and/or pose construction may for example be based on information obtained from the fiber optic sensor, e.g. in line with information as described in U.S. Pat. No. 7,813,599B2. Nevertheless, such methods and systems do not allow to take into account twist of the optic fiber sensor thereby obtaining an absolute shape and/or pose in an absolute reference system. By using methods and systems according to the present invention, such absolute shape and/or pose may be obtained.

The method also may comprise varying a parameter value in a parameter space for the shape and/or pose construction and determining, at the position of the second electromagnetic sensor, a minimum difference in position and/or orientation obtained from the second EM sensor and from the parameterized shape and/or pose construction. The latter typically will allow to determine a best suitable parameter value for the parameter. When for example only the position is used, the best parameter value may be the parameter for which the Euclidian distance between the position of the second electromagnetic sensor obtained from the position information measured with the second electromagnetic sensor and the corresponding position from the parameterized shape and/or pose construction is smallest.

The method furthermore comprises determining the shape and/or pose of the elongated element by inserting the determined best suitable parameter value in the parameterized shape and/or pose construction. Whereas the above exemplary method describes determining shape and/or pose of the elongated element for a single moment in time, the method also may be applied for subsequent moments in time. Information obtained in previous calculations can then in addition be used for finding the best suitable parameter values, taking into account that if changes occur in the parameter over time, such changes typically will be continuous and not discontinuous.

By way of illustration, embodiments of the present invention not being limited thereto, a first example illustrating features and characteristics of an embodiment of the present invention wherein two 5DOF EM-sensors are used and a single fiber is used is discussed below. In the present example, the EM sensors are fixed to the active shape sensing section but the fiber can rotate with respect to the two EM-sensors. The setup of the present example comprises an NDI Aurora field generator, mounted horizontally on a table so that the field of measurement is located in the vertical direction relative to the generator. A plexiglass cube and plate was placed on top of the generator to have a working space that is somewhere in the middle of the measurement volume in which the EM-sensors can be tracked. This measurement volume is defined by the electromagnetic field generator. On this plate, the fiber optic sensor is positioned in a tube connected to 2 EM-sensors by means of 2 plexiglass fixtures. They consist of a rectangular block that has two parallel holes: one for the tube and one for the EM-sensor. In this way, it ensures that the EM-sensor and fiber optic sensor are aligned. Tape is used to prevent the fixture to move over the tube. Since the fiber is free to rotate inside the tube, it can freely rotate or twist when the fixtures are moved.

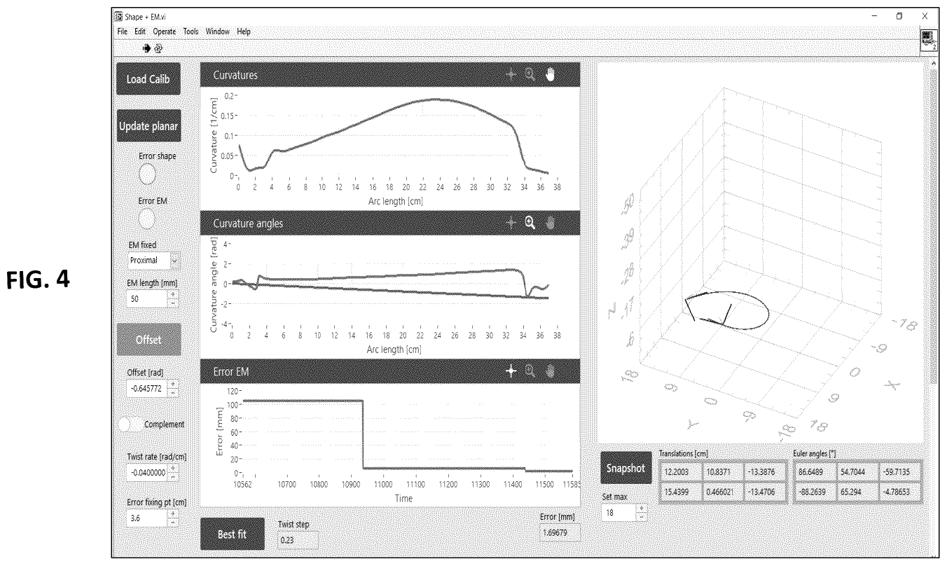

In the present example, the used sensors are actually 6DOF sensors but the rotation information (roll) was not being used. The fiber optic sensor has a length of 370 mm and contains 38 gratings that have a 10 mm physical spacing. The EM-sensor at the proximal end of the fiber (i.e. at the start of the sensing zone) was considered to be the first EM-sensor or the base EM sensor, i.e. its position and orientation are used to determine the origin and orientation for the fiber optic sensor at the start of the sensing zone. Dedicated software was made in LabVIEW that is capable to combine the data from the fiber interrogator and the EM-sensors. An example of a screenshot of the User Interface (UI) is shown in FIG. 4. The software makes a graphical representation of both EM-sensors and the optical fiber in 3D, together with the curvature and curvature angle (direction) of the fiber optic sensor.

The calibration file for the optical fiber can be loaded. It contains the strain sensitivity parameters and fiber configuration together with the nominal wavelengths (i.e. the wavelength in the absence of bending or strain) and information related to the intrinsic twist of the fiber (i.e. the twist that is inherently present in the glass structure after the fiber production).

The 2 fit parameters are the ‘Offset’, which represents the rotation at the base EM sensor, and the ‘Twist rate’. They can be entered manually via controls on the UI or can be optimized with a minimalization procedure via the ‘Best fit’ button. The offset performs a rotation of the shape around the tangential axis of the base EM-sensor. The twist rate adds a linearly increasing (or decreasing) rotation to the curvature angle as function of the fiber length and is represented by a red line in the curvature angle graph. The boundaries for the offset are from −π to π and for the twist rate from −0.12 rad/cm to +0.12 rad/cm (corresponding to almost ±2π/50 cm).

In order to evaluate the accuracy, the Euclidean distance between the second EM-Sensor and the optical fiber tip is calculated and indicated on the UI in the ‘Error EM’ graph and with the ‘Error [mm]’ indicator. Because the position between the second EM-sensor and the last grating is not exactly matching, the ‘Error fixing pt’ control is used to define the exact position of the second EM-sensor with respect to the last grating. The parameter indicates the arc length measured along the fiber from the last grating to the distal EM-sensor.

As a first step, the initial shape was put in a planar shape and the planar calibration was updated forcing the measured shape to be planar. The planar calibration actually compensates the curvature angle for ‘intrinsic twist’ that is present in the fiber. When the fiber is lying in a plane, the curvature angle should be constant. The planar calibration makes the curvature angle zero.

The EM sensor was positioned 2.6 cm from the last grating. The offset and twist rate were optimized with the best fit button to minimize the error. The best result was found for an offset of-1,0821 rad and a twist rate of zero. The zero twist rate was expected because of the planar calibration was done just prior to the measurement so the intrinsic twist was fully compensated. As a consequence, this best fit comes down to a pure rotation with respect to the base EM-sensor. The error with these updated parameters was found to be 0.98 mm demonstrating the accuracy of the shape sensing technology when twist is fully compensated.

To test the method, an out of plane movement of the fiber was applied. To do so, the fixture at the fiber tip was lifted and placed on a glass cup and pointing into a different direction. It resulted in a spiral-like shape. The resulting error without twist and rotation compensation was 64.7 mm. After the best fit procedure, the error reduced to 9.8 mm. Both rotation and twist rate were found to be non-zero. It should be noted that the step size for adapting the twist rate with the automatic minimalization procedure was set to 0.01 rad/cm. By manually adapting the twist rate with smaller values, the error could be further reduced.

In a further test, the effect of movement of the lead-in section is evaluated. This effect was created by flipping the fiber connector with 180°. This flipping of the connector results into a fiber rotation of 180° at the position of the connector as the fiber is fixed into the connector. The resulting error without twist and rotation compensation was about 135 mm. The error was slightly increasing even after the connector has been flipped. This can be explained by the fact that the induced rotation of the fiber at the connector side is slightly progressing through the capillary over time. The speed of this progression is predominantly determined by the friction between the fiber and the inner wall of the tube. After the best fit, the error is reduced to 6.1 mm. Both rotation and twist rate were found to be non-zero. Fact that a rotation correction is needed can be understood from the fact that a twist from the fiber (starting from the connector side) results into a rotation of the fiber at the position of the first FBG position corresponding in this case also to the base EM-sensor.

Further By way of illustration, embodiments of the present invention not being limited thereto, the following example illustrates features and advantages of particular embodiments of the present invention.

### ADDITIONAL FIRST EXAMPLE

In a first additional example, standard and optional features and advantages are shown, illustrating on how Advanced fibre optics shape sensing (FOSS) based reconstruction methods can be progressed to achieve advanced FOSS strategies for different purposes. The present example provides an illustration of dynamic twist estimation and compensation through EM data fusion. This strategy is introduced in the following sections.

In state of the art methods there are still several limitations to be identified. Calibration of the different sensing modalities in a practical setting is often complex and time-consuming. Moreover, calibrations may be impractical since there are no lead markers in a real patient's anatomy. Accordingly, other methods have to be followed, which would most likely have an effect on the final registration accuracy.

The present example, the focus is on employing FOSS technology solely to facilitate instrument visualization and provide information regarding the instrument's progression with respect to a defined reference. A problem that is overcome, however, is that the FOSS-based reconstruction of a three-dimensional spatial curve Cs is represented with respect to a local reference frame defined by the MCF's TNB frame (or alternatively the TM1M2 frame). The choice of this local frame is often arbitrary and remains constant regardless of the FOSS configuration. An example would be to choose a [3×3] identity matrix to define the TNB frame at the base of the reconstructed curve. This means that at the location of the base coordinate (for any and all reconstructed curves), the tangent vector T would point in the local x-axis direction, the normal vector N in the local y-axis direction, and the binormal vector B in the local z-axis direction. The reconstructed curve would always be initiated with this frame representation, and the location of the base coordinate would always be at a fixed position. This would be the case even though the instrument's actual position is dynamically changing.

To this end, consider a scenario where an instrument with embedded FOSS would be navigated through a patient's vasculature. Here, the reconstructed curve would normally be represented with respect to its own reference frame, i.e. the local frame of the fiber {f}. This would not provide the necessary useful information about how the instrument changes its shape with respect to its surrounding environment, i.e. with respect to a global frame {g}. In other words, while it may still be useful to have a visualization of the instantaneous instrument shape, having it represented with respect to a known and global reference frame {g} would provide more useful information about its location and interactions. This global reference should be easily visualized by the interventionalist.

The proposed method in this example reconstructs the complete shape of the instrument, but it can also provide real-time 6-DoF pose tracking of any point along its curve. Compared with EM tracking, FOSS can provide extra information, namely: (1) the complete three-dimensional reconstructed curve representing the instrument's shape, and (2) the 6-DoF pose of any point along the curve including the tip.

One of the main factors that affects the shape reconstruction accuracy is dynamic twist. Twist may be the most crucial factor that has the largest impact on shape accuracy. In the present example the concept of sensor fusion is utilized here to provide an estimate of the magnitude of dynamic twist subjected to the MCF. From this knowledge, it then becomes possible to compensate for twist in an improved shape reconstruction algorithm. The approach proposed in this example thus makes use of data fusion between FOSS and EM sensor technology to achieve dynamic twist estimation and compensation.

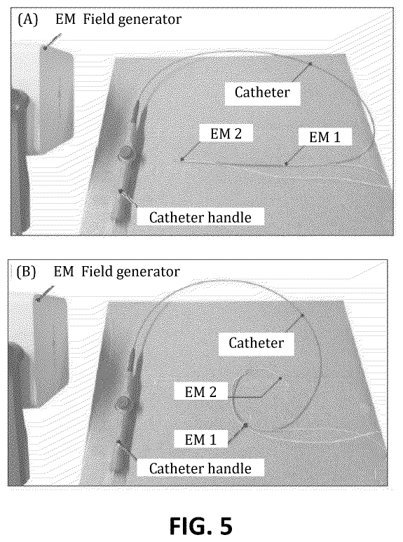

Now EM data fusion for dynamic twist compensation by integrating two EM sensors onto the catheter body as used in the present example is discussed. One EM sensor is rigidly fixed to the location of the first proximal FBG, while the second EM sensor is rigidly fixed to the location of the last distal FBG. This is illustrated in the experimental setup of FIG. 5. For calibration simplicity, the z-axis of the proximal EM sensor (i.e. the sensor's longitudinal axis) is made parallel with the longitudinal axis of the catheter. Accordingly, it can then be assumed that the EM's z-axis is parallel with the tangent vector T at the base. This EM configuration is not strictly required, but merely simplifies the calibration. The proximal EM sensor frame is considered as the global frame, such that the frame of the distal EM sensor is represented with respect to it. Rigidly fixing the proximal EM sensor to the location of the first FBG means in the present example that there is no required translation between the EM frame and the reconstructed shape reference frame. There will be a slight displacement along the radial direction of the catheter, but this is considered to be negligible here. On the other hand, the relative orientation between the proximal EM sensor frame and the reference shape reconstruction frame must be found. This can be achieved through measurement of experimental data. The catheter with the embedded MCF and EM sensors is made into a curved configuration and placed onto a planar surface as shown in FIG. 5. The amount of curvature subjected to the catheter is then varied. This is done while the catheter remains strictly along the plane. The reason for this strict planar requirement is to minimize and limit the effect of dynamic twist. As long as the catheter is under a plane, static (or intrinsic) twist compensation is assumed to be sufficient to obtain adequate shape reconstruction accuracy. The MCF wavelength data and the EM pose data are measured and recorded simultaneously during this calibration process. The rotation matrix

$${}_{{C}_{s}}^{EM} R ,$$

between the reference reconstructed frame and the proximal EM sensor frame can be found from the measured data through singular value decomposition (SVD) or optimization through least squares minimization. The objective cost function C(Θ) to be minimized in this case would be:

$$C ( Θ ) = {\sum }_{i = 1}^{k} ‖ {P}_{EM , k} - {P}_{tip , k} ‖ \qquad [ 1 ]$$

$$for C ( Θ ) subject to$$

$$\{ \begin{matrix}{C}_{s 2 , k} = {}_{{C}_{s}}^{EM} R ( Θ ) {C}_{s , k} \\ {P}_{tip , k} = {C}_{s 2 , k} ( s = l )\end{matrix}$$

where Θ is the [3×1] optimization parameters vector containing the roll, pitch, and yaw angles defining

$${}_{{C}_{s}}^{EM} R ,$$

k is the index of the current time step, p_{EM,k} is the three dimensional position vector of the distal EM sensor, and p_{tip,k} is the three dimensional position vector of the tip coordinate of the reconstructed curve after multiplying it with

$${}_{{C}_{s}}^{EM} R ,$$

(which is defined by Θ). Finally, the obtained rotation matrix^{EM} R minimizing the objective function is then used to align the reference reconstructed curve's frame with the frame of the proximal EM sensor.

Twist subjected to the MCF body results in an inaccurate estimation of the bend angle θ_{b}. That is because twist causes the MCF's material frame to rotate about its longitudinal axis, thus affecting the bend angle θ_{b} but not the curvature κ. Given the axisymmetric and homogeneous nature of the MCF, twist is assumed here to increase linearly (i.e. at a constant twist rate) from the location of the first most proximal FBG towards the last most distal FBG. Considering a MCF with m FBG sets along its length, dynamic twist ϕ(j) at the location of each corresponding FBG set would be given as:

$$ϕ ( j ) = \frac{j}{m - 1} {ϕ}_{max} \qquad [ 2 ]$$

where j=[0, 1, 2, . . . , (m−1)] represents the index of a given FBG set, and ϕₘₐₓ is the magnitude of angular twist at the location of the most distal FBG, i.e. at j=m−1. Note that expression [2] however, assumes that the most proximal FBG is rigidly fixed and is subjected to no twist. This is a rather limiting assumption that is only valid in configurations where indeed the most proximal FBG is rigidly fixed to its enclosing structure. The MCF is rather typically fixed to the catheter at the entry port near the catheter handle. If twist is present throughout the catheter body, then the first most proximal FBG would also be subjected to twist. Accordingly, expression [2] can be adapted to include this behaviour by adding a twist offset ϕ_{off} such that:

$$ϕ ( j ) = {ϕ}_{off} + \frac{j}{m - 1} {ϕ}_{max} \qquad [ 3 ]$$

If we consider θ_{b}(j) to be the measured bend angle at the position of the j^{th} FBG set, then the twist incorporating bend angle θ_{b.t.w}(j) would be given as:

$${θ}_{b , tw} ( j ) = {θ}_{b} ( j ) + ϕ ( j ) \qquad [ 4 ]$$

The effect of dynamic twist on the bend angle could thus be compensated by estimating the two parameters ϕ_{off} and ϕₘₐₓ. The integration of the EM sensors is used for this estimation purpose. Here, least squares optimization can be employed to estimate the aforementioned two parameters by minimizing the following objective cost function C(Θ):

$$C ( Θ ) = ‖ {P}_{EM , k} - {P}_{tip , k} ‖ \qquad [ 5 ]$$

where Θ is the optimization parameters vector containing ϕ_{off} and ϕₘₐₓ, k is the index of the current time step, p_{EM,k} is the three dimensional position vector of the distal EM sensor, and p_{tip,k} is the three dimensional position vector of the tip coordinate of the reconstructed curve represented in the same reference frame as the proximal EM sensor. It is to be noted that the optimisation in some embodiments also may be performed for a combination of time steps, e.g. where the twist and rotation are considered to be constant. In such a case, the optimisation may be determined using summation over the set of time steps:

$$C ( Θ ) = {\sum }_{i = 1}^{k} ‖ {P}_{EM , k} - {P}_{tip , k} ‖$$

The EM sensors are utilized to provide information about the curve's proximal and distal points which is then used to estimate the twist parameters ϕ_{off} and ϕₘₐₓ and to rectify the reconstructed shape such that the position of the reconstructed curve tip matches the position of the distal EM sensor.

The experimental setup shown in FIG. 5 was used to validate the performance of the proposed EM data fusion strategy. The initial step was to find the rotation matrix

$${}_{{C}_{s}}^{EM} R ,$$

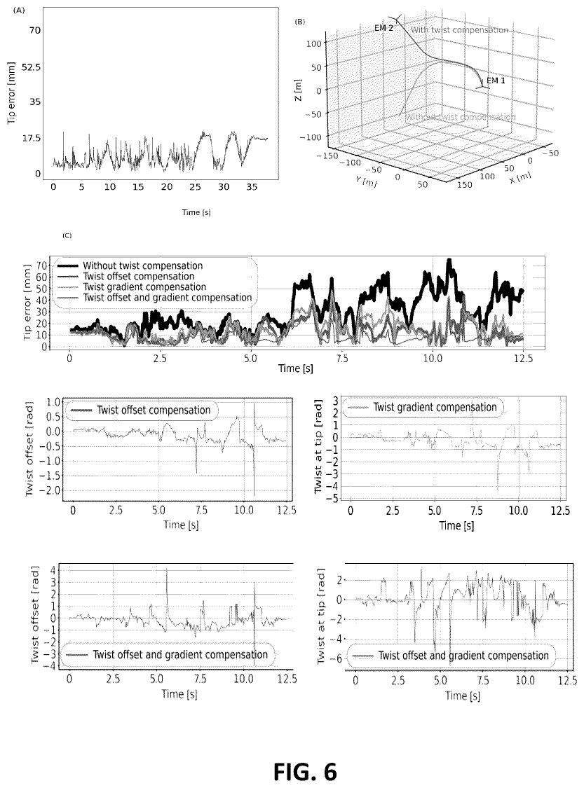

between the reconstructed curve's reference frame and the frame of the proximal EM sensor, as described above. The resulting distance errors between the position of the distal EM sensor and the tip of the reconstructed curve after this initial step are shown in FIG. 6 part (A). The mean and maximum errors were found to be 9.3±5.8 mm and 22. 1 mm, respectively. The reason for this relatively large tip error is most likely due to the assumption of having the z-axis of the proximal EM sensor being parallel to the tangent T vector of the reconstructed curve. In practice, however, this is not exactly true. Nevertheless, the exact magnitude is not the most relevant quantity to be analysed here, but rather the improvements coming from the EM data fusion strategy. Accordingly, the obtained mean and maximum errors in this calibration configuration will be taken as the reference when comparing the other configurations.

The next step was to manipulate the catheter in three-dimensional space while imposing a diversity of curvature and torsion profiles. This was done for a total duration of around 12.5 seconds. In this experiment, the total interrogated length of the MCF was 22.2 cm. To illustrate the importance of estimating both twist parameters ϕ_{off} and ϕₘₐₓ, the EM data fusion strategy was carried out by incorporating: (1) ϕ_{off} only, (2) ϕₘₐₓ only, and (3) both ϕ_{off} and ϕₘₐₓ in the twist compensation model. The results of this experiment are depicted in FIG. 6 part (B) and FIG. 6 part (C). The tip errors for those three different strategies in addition to the tip errors without twist compensation are summarized in the following table.

TABLE
Experimental distance errors between EM sensor position
and reconstructed shape tip for different compensation
strategies (all dimensions are in millimetre).

| | No | Offset | Gradient | Offset and |
|---|---|---|---|---|
| | comp. | comp. | comp. | gradient comp. |
| Mean error | 28.6 | 12.3 | 14.3 | 8.0 |
| Std. error | 16.0 | 7.5 | 9.1 | 5.7 |
| Maximum | 75.1 | 43.3 | 48.3 | 40.8 |
| error | | | | |

The results illustrate notable improvement in tip errors for all three strategies. This is a logical result since the twist contribution is composed of both the twist offset ϕ_{off} and the twist gradient defined by ϕₘₐₓ. However, the most notable improvement in the reconstructed shape occurs when both twist parameters are incorporated. The mean error drops from 28.6 mm to 8.0 mm, which is a 72.0% improvement. The relevancy of compensating for dynamic twist can be clearly observed from FIG. 6 part (B). Here the reconstructed shapes with and without twist compensation are depicted. The large difference between the two shapes is clear, and proves the necessity for twist estimation and compensation for improved shape reconstruction accuracy.

In the present example, aspects related to advanced FOSS-based strategies were shown focusing on three main topics, which were namely: (1) curvature-based shape registration, (2) instrument tip pose tracking, and (3) EM data fusion for dynamic twist compensation.

The first of these topics discussed how additional information about the FOSS-based reconstruction can be extracted by employing a simple predefined shape. In practical interventions, this means that the operator would be able to know the insertion length of the catheter and have catheter shape feedback being provided with respect to a known and visible reference. This makes the catheter visualization process much easier and facilitates the intervention improving safety and efficacy.

The second topic discussed how FOSS-based reconstruction could be used for instrument tip pose tracking. The overall method to achieve this objective is carried out by comparing the reconstructed shape at any given time instant with a reference shape. Consequently, the pose of any point on the reconstructed curve can be defined with respect to this reference. Experimental validation of the method compared with conventional EM sensing demonstrated the accuracy and efficacy of the proposed method. This was clearly illustrated by the high correspondence between the tip motion profile reconstruction for both methods (mean error of 0.16±0.60 mm). This is an interesting result since FBG-MCFs have a multitude of advantages when compared to other sensing modalities. Furthermore, FBG-MCFs can be simultaneously employed for several of other purposes, e.g. catheter tip force sensing or body force estimation. FOSS-based instrument tip tracking could thus be a viable, and potentially superior, alternative to present-day methods.

Finally, the third topic elaborated on the importance of twist estimation and the method to achieve this objective employing EM sensor technology. The benefits provided by fusing EM data on the resulting shape reconstruction accuracy are evident. Moreover, it is important to note that this is a practically feasible option since many commercial catheters today already incorporate such EM sensors within their embodiments (e.g. the Thermocool SmartTouch® ablation catheter from Biosense Webster). The method illustrated how an optimization problem can be formulated to match the tip position of the reconstructed shape to the position of the distal EM sensor. Note that the EM orientation can also be optionally included in the optimization problem when estimating ϕ_{off} and ϕₘₐₓ to constrain the complete pose of the reconstructed curve's tip to the one of the distal EM sensor. This was not included here given that the motivation of this work is to prove the principles of EM data fusion only and to maintain calibration simplicity.

### ADDITIONAL SECOND EXAMPLE

In the second additional example, an approach for tracking the catheter is illustrated to address the issue of the highest shape sensing error at the tip and effectively utilize data from EMT sensors to enhance the accuracy of the optical-based shape sensing method. The catheter shape is directly reconstructed in the EMT coordinate frame by approximating the catheter shape by a number of Bézier curves while taking into account the curvatures measured by the optical fiber. Both 2D and 3D shape sensing experiments were done to verify features and advantages of the present exemplary embodiment. The results of the 3D experiment show that the proposed method reduces the mean catheter shape tracking error by approximately 38% (from 12.1 mm to 5.4 mm for a sensed length of 540 mm long) compared to the traditional method where the same number of sensors were used. The present example illustrates a catheter tracking approach based on a combination of EMT sensors and a multi-core FBG fiber. The proposed approach allows incorporating the information from the EMT sensors in the 3D shape reconstruction process itself. Unlike the traditional approach, the 3D catheter shape will in the present example be approximated by multiple Bézier curves. Compared to the state-of-the-art EMT sensors-based shape sensing methods, the Bézier curve fitting process not only uses the pose information from the EMT sensors but also uses the curvature measured by the FBG fiber. This approach aids in reducing the number of integrated EMT sensors. The problem of error build-up by integrating the errors over the measurement length in the classic FBG-based shape sensing method is now resolved since the tip point and other points along the catheter length are now constrained by information provided by the EMT sensors. An advantage of the proposed exemplary method is that the EMT sensors information is now, not only to localize the reconstructed shape in a global coordinate frame but also helps improving the estimated shape.

A further advantage of the proposed fusion approach is that without explicit modeling of the dynamic twist, the effect of dynamic twist acting on the straight configuration multi-core fiber is also compensated. It is worth noting that although the multi-core FBG fiber is used here to demonstrate the proposed approach, the approach can be easily transferred to other fiber optic-based shape sensing techniques (in both straight and helical configurations). A set of 2D and 3D experiments are done to demonstrate the value of the proposed approach.

The traditional EMT and FBG-based and EMT-based catheter tracking methods are also implemented and serve as a baseline against which the newly proposed approach is compared to.

The experimental results that the new estimation method outperforms the traditional approach in 2D and 3D experiments.

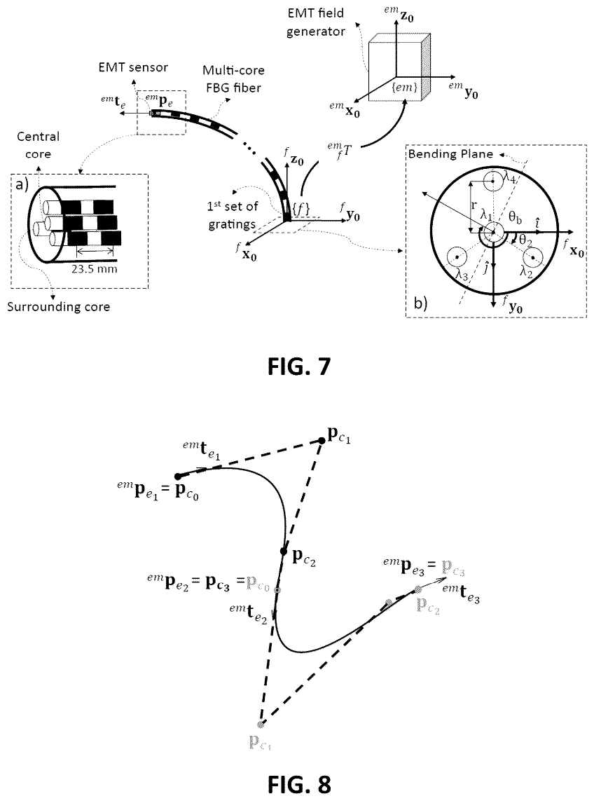

FBG-based shape sensing methods rely on measured discrete curvatures and bending plane angles along the length of a fiber. These curvatures are obtained by employing an interrogator to monitor the change in the reflected wavelength of each grating. A method to calculate the curvature and the angle of the bending plane from the measuring wavelength shift is described below. In the traditional approach, the 3D shape of the catheter in the FBG local coordinate frame {f} is then reconstructed. Afterward, the 3D shape is localized in the fixed EMT coordinate frame {em} by using additional information from the EMT sensors. The relation between {f} and {em} coordinate frames is described in FIG. 7. The traditional 3D shape reconstruction and localization process are briefly summarized below, whereas the newly proposed catheter tracking approach is detailed.

FIG. 7 parts (a) and (b) depict the construction of a multi-core FBG fiber. A multi-core FBG fiber commonly has four cores. The first core is located in the center of the fiber and it aligns with the neutral axis of the fiber. Then, there are three surrounding cores at a distance of r from the central core. These cores are spaced 120° rotated around the central core. Each core features the same number of gratings distributed along its length. Each grating is a Bragg reflector that reflects a particular wavelength of the incoming light while transmitting all others. The central wavelength of the reflected light of each grating is named the Bragg wavelength λ_{B}. The reflected wavelengths are measured by an interrogator connected to the proximal end of the multi-core FBG fiber. The wavelength of each grating varies depending on the change of the surrounding temperature AT and the applied mechanical strain ε. The measured wavelength shift is given by

$$\frac{{λ}_{B} - {λ}_{{B}_{0}}}{{λ}_{{B}_{0}}} = \frac{Δλ}{{λ}_{{B}_{0}}} = {S}_{ε} ε + {S}_{T} Δ T$$

where S_{ε} and S_{T} are the strain and temperature sensitivity coefficients of the fiber, respectively. λ_{B0} is the unstrained Bragg wavelength of the grating. The gratings in the central core are only sensitive to axial strain and temperature variations. When the axial strain is negligible, as is commonly the case with catheters, the wavelength changes due to temperature variations can then be computed from this central core. The bending strain applied on the outer cores ε_{BendiE{2,3,4}} can subsequently be determined as follows

$${ε}_{{Bend}_{i ∈ \{ 2 , 3 , 4 \}}} = \frac{Δ {λ}_{i}}{{λ}_{{B}_{0 i}} {S}_{ε}} - \frac{{Δλ}_{1}}{{λ}_{{B}_{01}} {S}_{ε}}$$

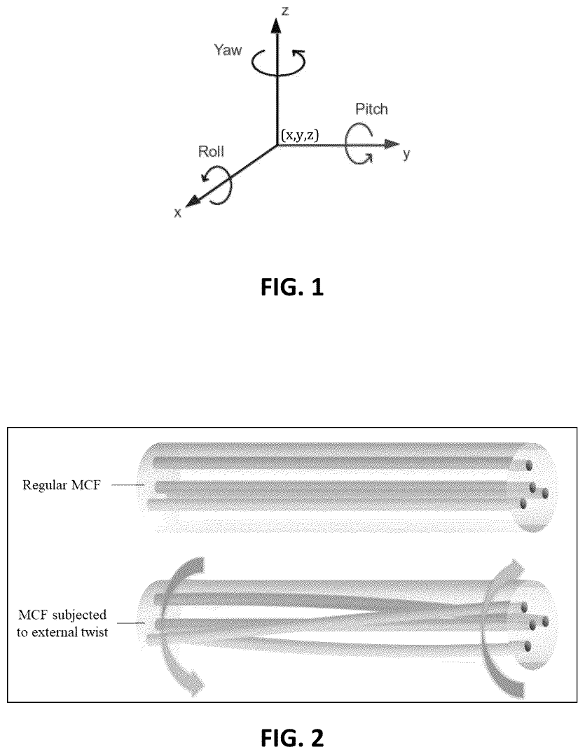

As such the multi-core fiber allows measuring three bend induced strains per cross-section where a set of gratings is present. These strains can then be used to calculate the curvature κ_{EBG} and the angle of the bending plane θ_{bFBG} at each measurement cross-section. The relation between the bend induced strain, the curvature and the angle of the bending plane is given by

$${ε}_{{Bend}_{i ∈ \{ 2 , 3 , 4 \}}} = - {κ}_{FBG} r sin ( {θ}_{{b}_{FBG}} - \frac{3 π}{2} - {θ}_{i} )$$

where r is the distance from the outer cores to the central core; θi is the angle of the ith outer core and θbFBG the angle of the bending plane corresponding to the x-axis of the fiber (as shown in FIG. 1 part (b). A closed-form solution is given by:

$${κ}_{app} = {\sum }_{i = 1}^{3} \frac{{ε}_{{Bend}_{i}}}{r} cos {θ}_{i} \hat{i} - {\sum }_{i = 1}^{3} \frac{{ε}_{{Bend}_{i}}}{r} sin {θ}_{i} \hat{j}$$

$${κ}_{FBG} = \frac{2 \vert {κ}_{app} \vert}{3}$$

$${θ}_{{b}_{FBG}} = {∠κ}_{app} ,$$

where î and ĵ are the unit vectors along the x- and y-axes of the fiber's cross section, respectively, as depicted in FIG. 7 part (b).

A traditional FBG-based catheter tracking approach includes two main steps. Firstly, the catheter shape is estimated in the local coordinate frame that is defined here, without loss of generality, to coincide with the most proximal grating of the fiber. The 3D reconstructed shape of the catheter is then localized in the global EMT coordinate frame by exploiting the information provided by the attached EMT sensors. The 3D catheter shape estimation process based on the discrete curvatures and the angles of the bending plane is briefly discussed below. The catheter shape localization process is also described.

Traditional FBG-based catheter shape estimation: The set of discrete curvatures and the angles of the bending plane are first interpolated to improve the estimated shape and maintain a quasi-continuous curvature profile along the catheter's arc length. Assuming that the entire arc length of the catheter is discretized into u points, the sets of the interpolated curvatures and angles of bending plane are denoted as κ_{FBGint} and θ_{bFBGint}, respectively. A continuous and differentiable space curve can be used to represent the shape of the catheter. This space curve is defined by curvature κ(s) and torsion τ(s) profiles with the arc length variable s that changes from s=1 at the first set of grating to s=u at the last set of grating. The torsion τ(s) is the rate of change of the angle of the bending plane θ_{b} along the fiber length. The interpolated curvature and torsion profiles define how the tangent t, normal n, and binormal b unit vectors (TNB frame) evolve along the arc length. The differential Frenet-Serret formula can be used to solve for the evolution of the moving TNB frame. The position c(s) of each point along the catheter shape can then be calculated by integrating the tangent unit vectors as follows

$${}^{f} c ( s ) = {}^{f} c ( 1 ) + {\int }_{1}^{s} t ( v ) dv$$

where f_{c}(1) is the position of the catheter's base.

Catheter shape localization in EMT coordinate frame:

To localize the reconstructed 3D shape of the catheter in a fixed EMT coordinate frame {em}, a spatial calibration step needs to be done in advance. Since the relative pose of {f} versus {em}

may vary due to manufacturing. The following calibration procedure can be conducted to retrieve this information. First, the catheter is fixed at the level of the most proximal EMT sensor. It is then bent in planar in two configurations symmetric with respect to the straight configuration. The distance between the first and second configuration of each corresponding point at a given arc length along the 3D reconstructed shape is computed. The travel distance of each point along the length of the reconstructed shape is then compared to the travel distance of each distal EMT sensor. This allows for devising the corresponding arc length where each EMT sensor is located.

After that, the distance between the most proximal sensor to the distal EMT sensors is calculated. This can be done when the catheter is in a straight configuration. From the distance between the proximal and the distal EMT sensors in the straight configuration and the correspondence between the distal EMT sensors to the 3D shape reconstructed shape, the correspondence of the proximal sensor can then be estimated.

Assuming that m EMT sensors are attached to the catheter and the pose measured by each EMT sensor corresponds to the pose of a point along the arc length of the FBG-based 3D reconstructed shape

$${}^{f} c ( {s}_{{EMT}_{i}} )$$

where i={1, . . . , m}, the 3D shape of the catheter can then be localized in the EMT coordinate frame. The correspondence arc length of the ith EMT sensor s_{EMT}_{ᵢ} is estimated by the above-mentioned spatial calibration step described in the previous part. Each EMT sensor provides its location

$${}_{}^{em} {p}_{{e}_{i}}^{}$$

and its unit tangent vector

$${}_{}^{em} {t}_{{e}_{i}}^{}$$

in the EMT coordinate frame. A set of poses (including position and tangent vector) provided by the EMT sensors in {em} and their correspondences in {f} can be used to obtain the transformation matrix

$${}_{f}^{em} T$$

e.g. by using a point-to-point registration method. This transformation matrix then transforms the reconstructed shape from the local frame {f} to the fixed EMT coordinate frame {em}. The catheter shape in the EMT coordinate frame can be obtained by

$${}^{em} c ( s ) = {}_{f}^{em} {T}_{}^{f} e ( s )$$

The traditional method presented in the previous section requires first reconstructing the shape. The EMT sensors are only used to find the transformation matrix to map the reconstructed shape to the EMT frame. In the newly proposed approach, the catheter shape is directly reconstructed in the EMT coordinate frame in a single step. The proposed approach uses the information from the EMT sensors not only to localize the catheter shape but also to improve the shape estimation. The catheter shape is approximated by multiple Bézier curves. A Bézier curve of degree n is specified by n+1 control points and is defined as

$$b ( t ) = {\sum }_{i = 0}^{n} ( \begin{matrix}n \\ i\end{matrix} ) {( 1 - t )}^{n - i} {t}^{i} {p}_{{c}_{i}}$$

$$where ( \begin{matrix}n \\ i\end{matrix} )$$

are the binomial coefficients and t∈[0;1]; p_{c}_{ᵢ} are the control points. The Bézier curve starts at p_{c}_{₀} and ends at p_{c}_{_{n′}}. The two control points p_{c}_{₁} and p_{c}_{ₙ₋₁} lie along tangent vectors at the starting and the end point.

Assuming that m EMT sensors spaced at regular intervals are included along the length of the catheter, the catheter shape between two consecutive j^{th} and j+1^{th} EMT sensors can then be approximated by a Bézier curve. The first control point p_{c}_{₀} and the last control point p_{c}_{ₙ} of the Bézier curve between two subsequent EMT sensors can be defined by

$${}_{}^{em} {p}_{{e}_{j}}^{} and {}_{}^{em} {p}_{{e}_{j + 1}}^{} ,$$

respectively. An optimization problem can then be formulated to find the remaining control points (p_{c}_{₁}, . . . , p_{c}_{ₙ₋₁}) by minimizing the cost function

$${arg min}_{x = {p}_{{c}_{1}} , … , {p}_{{c}_{n - 1}}} α {E}_{length} + β {E}_{κ}$$

where Elength is the error in the length of the estimated Bézier curve. Theoretically, the length of the estimated Bézier curve would be equal to the arc length between two consecutive EMT sensors j^{th} and j+1^{th} (denoted as l_{Straight}). The arc length l_{straight} can be measured by putting the catheter in a straight configuration. The length error Elength is calculated as:

$${E}_{length} = {( {l}_{Straight} - {l}_{Estimate} )}^{2}$$

The arc length of the estimated B'ezier curve can be derived analytically or via numerical integration. The error in curvature Eκ is the difference between the curvature along the length of the estimated Bézier curve κB and the curvatures obtained from the FBG sensors over the corresponding section κ_{FBG}_{ᵢₙₜ} (s=s_{EMT}_{ⱼ}, . . . , s_{EMT}_{ⱼ₊₁}). Given that the estimated Bézier curve is discretized into ub points (u_{b}=s_{EMT}_{ⱼ₊₁}−s_{EMT}_{ⱼ}), the error in curvature Eκ between the j^{th} and j+1^{th} EMT sensor can be computed as:

$${E}_{κ} = {\sum }_{s = {s}_{{EMT}_{j}}}^{{s}_{{EMT}_{j + 1}}} {( {κ}_{{FBG}_{int}} ( s ) - {κ}_{B} ( t ) )}^{2}$$

$$where t = \frac{s - {s}_{{EMT}_{j}}}{{u}_{b}} .$$

The curvature κB(t) of a parameterized curve can be calculated as:

$${κ}_{B} ( t ) = \frac{‖ {b}^{′} ( t ) \times {b}^{″} ( t ) ‖}{{‖ {b}^{′} ( t ) ‖}^{3}}$$

The scaling factors a and B regulate the relative weight between E_{length} and Eκ. The optimization problem strives to identify the control points of a Bézier curve that aligns in curvature with the curvatures measured by the optical fiber and has the same length as the arc distance between two consecutive EMT sensors.

In case the shape between two consecutive EMT sensors is approximated by a Bézier curve of degree n, n−1 control points need to be solved. Each control point is defined by three scalar values. Thus 3×(n−1) variables are searched for in the optimization problems. Since p_{c}_{₁} and p_{c}_{ₙ₋₁} are the two control points that lie along tangent vectors at the starting and end point, the set of optimization variables for a Bézier curve of degree n is now reduced to 3×(n−1)−4 elements x={lₛₜₐᵣₜ, l_{end}, p_{c}_{₂}, . . . , p_{c}_{ₙ₋₂}}. Then, p_{c}_{₁} and p_{c}_{ₙ₋₁} can then be calculated as:

$${p}_{{c}_{1}} = {p}_{{c}_{0}} + {l}_{start} {}_{}^{em} {t}_{{e}_{j}}^{}$$

$${p}_{{c}_{n - 1}} = {p}_{{c}_{n}} - {l}_{end} {}_{}^{em} {t}_{{e}_{j + 1}}^{}$$

An example of fitting the catheter shape by 3rd order Bézier curves is shown in FIG. 8. The choice of the degree of the Bézier curve depends on the complexity of the shape that the catheter can take on between two consecutive EMT sensors. The higher degree Bézier curve can represent more complex shapes. However, as the Bezier curve's degree increases, there are more control points that the optimization problem solver must find. For this reason, this work investigates only 3rd and 4th order Bézier curves.

Different solvers can be used to tackle this optimization problem such as the trust-region reflective or Levenberg-Marquardt. Most of the solvers accept an initial guess for the value of each optimization parameter. The quality of the initial guess impacts the ability to converge to a global or local minimum and the speed of the convergence. To have a good initial guess for the missing control points, it is proposed here to use the traditional approach as a first guess to estimate the catheter shape in the EMT coordinate frame. The shape between two consecutive EMT sensors provided by the traditional approach is approximated by a Bézier curve. The control points of the approximated Bézier curve can then be used as the initial guess for the optimization problem solver. To approximate a segment by a Bézier curve, the matrix form of the Bézier curve, based on the control points, can be used. The control points of 3rd and 4th order Bézier curves can be approximated by

$$[ \begin{matrix}{p}_{{c}_{0}} \\ {p}_{{c}_{1}} \\ {p}_{{c}_{2}} \\ {p}_{{c}_{3}}\end{matrix} ] = {( [ \begin{matrix}{t}^{3} & {t}^{2} & t & 1\end{matrix} ] [ \begin{matrix}- 1 & 3 & - 3 & 1 \\ 3 & - 6 & 3 & 0 \\ - 3 & 3 & 0 & 0 \\ 1 & 0 & 0 & 0\end{matrix} ] )}^{+} {}^{em} c$$

$$and$$

$$[ \begin{matrix}{p}_{{c}_{0}} \\ {p}_{{c}_{1}} \\ {p}_{{c}_{2}} \\ {p}_{{c}_{3}} \\ {p}_{{c}_{4}}\end{matrix} ] = {( [ \begin{matrix}{t}^{4} & {t}^{3} & {t}^{2} & t & 1\end{matrix} ] [ \begin{matrix}- 1 & - 4 & 6 & - 4 & 1 \\ - 4 & 12 & - 12 & 4 & 0 \\ 6 & - 12 & 6 & 0 & 0 \\ - 4 & 4 & 0 & 0 & 0 \\ 1 & 0 & 0 & 0 & 0\end{matrix} ] )}^{+} {}^{em} c$$

respectively where t=1 . . . u_{b}]^{T}. The matrix ^{em}c is a u_{b}×3 matrix that contains the catheter shape in {em} (corresponding to the arc length s=s_{EMT}_{ⱼ} NO s_{EMT}_{ⱼ₊₁}) estimated by the traditional method.

Both 2D and 3D experiments were performed to verify the proposed method. In the 2D experiment, optical markers were used with a camera to generate ground truth shapes. An advantage of the 2D experiment is that it allows examining the accuracy of our proposed catheter tracking method in different cases (using a different numbers of integrated simulated EMT sensors) with minimal effort in preparing different catheters at this stage. This is only feasible in the 2D experiment since each optical marker offers a location as well as a tangent vector. However, in practical situations, the catheter is often bent into 3D shapes. Due to this reason, 3D experiment was also performed. Unlike the 2D experiment, ground truth in the 3D experiment was acquired by fiducials attached along the fiber length tracked by a 3D real-time optical tracking system.

### 2D Experiment:

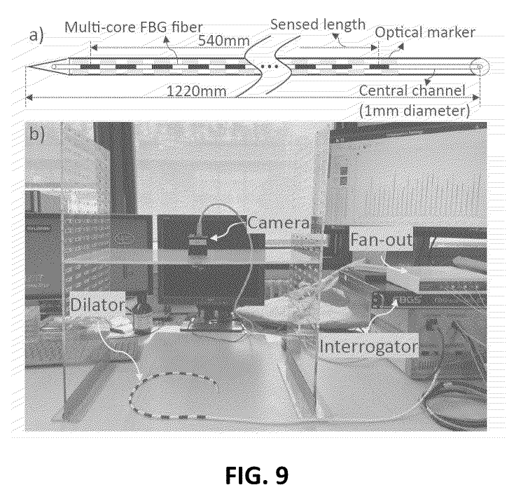

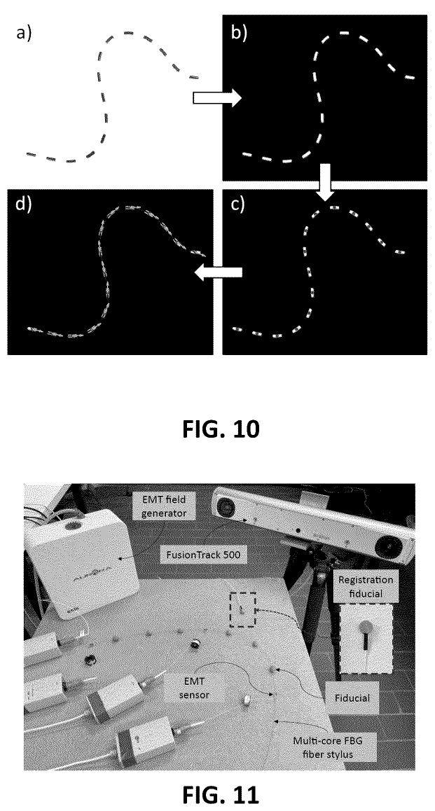

To verify the proposed catheter tracking approach and compare with the traditional FBG-based catheter tracking method, a dilator (Abbott, USA) with an embedded multi-core FBG fiber (FBGS, Geel, Belgium) in the central channel has been prepared. The schematic of the sensorized dilator is shown in FIG. 9 part (a). The fiber includes 4 cores. Each core features 24 gratings with a spacing of 23.5 mm between each set of gratings. With this configuration, the fiber is able to sense a 540 mm long shape. There are 15 optical markers attached along the dilator's length with a spacing of 20 mm. In the 2D experiment, the dilator is bent into different shapes on the 2D plane. An overhead monocular camera (Prosilica, Allied Vision Technology, Germany) is positioned above the setup, facing downwards, to capture the dilator's shape. The recognized shapes in the images are used as the ground truth. The dilator is supported on a plexiglass plate to ensure that the bending is in a plane parallel to the image plane. An interrogator (FBGscan 908 EP) from FBGS is used to record the wavelength shifts during the experiments. The experimental setup for 2D experiments can be seen in FIG. 9 part (b). Since the dilator only experiences in-plane bending, it is straightforward to use optical markers to simulate the EMT sensors. In the first case, 3 optical markers (the 1st, 8th and 15th marker) are used to simulate EMT sensors and to reconstruct the shape of the dilator while in the second case, 4 optical markers (the 1st, 5th, 10th and 15th marker) are used. In both cases, the shapes between 2 consecutive optical markers are approximated by 3rd and 4th order Bézier curves. Simple color segmentation is used to recognize the optical markers in the image frame. The position of the optical markers is defined as the centroid of the contour of each marker. The ground truth shape is obtained by fitting a B-spline curve to the set of positions of all optical markers. The tangent vector of each marker is calculated by taking the first derivative of the fitted B-spline curve. A known-size checker board is used to find the scale factor that allows transforming the recognized ground truth shape from pixel to mm scale. A sequence of the ground truth generation process is visualized in FIG. 10.

Using the measured wavelength shift and the marker poses, the traditional and the proposed catheter tracking approaches are applied to estimate the shape of the dilator in the image coordinate frame. The EMT-based shape sensing method presented in previous work [5] is also implemented to estimate the catheter shape. The performance of the three methods is compared via shape estimation error for each dilator's configuration. The shape estimation error is calculated by the mean and max distance between each point along the length of the estimated shape to the closest point from the ground truth shape. The closest points between two sets of points can be found by using the MATLAB (The MathWorks, Inc., Massachusetts, United States) function dsearchn.

### 3D Experiment:

In the 3D experiment, a multi-core FBG stylus made by FBGS has been used. The stylus includes a multi-core FBG fiber inserted into a Nitinol tube. The fiber contains 4 cores each core includes 39 gratings with a spacing of 14 mm. This multi-core fiber allows measuring a shape of 532 mm long. Three EMT sensors are attached to the stylus at approximately the 1st, 20th and 39th grating by means of a 3D printed fixture. The stylus has 6 spherical fiducials spaced evenly over its length. The 3D positions of these spherical fiducials can be tracked by a real-time optical pose-tracking system—fusionTrack 500 (Atracsys, Puidoux, Switzerland). The 3D experiment setup is described in FIG. 11. In this experiment, the stylus is bent into different configurations.

The 3D shape of the stylus is reconstructed in the EMT coordinate frame by both the traditional and the proposed method using information provided by the FBG fiber and the 3 EMT sensors. The 3D positions of the fiducials recognized by the fusionTrack system (accuracy of 0.09 mm) will be used as ground truth. The EMT coordinate frame and the fusionTrack coordinate frame need to be registered in advance. To register these two coordinate systems, a registration fiducial has been prepared. This registration fiducial features an EMT sensor inserted into the central channel of the spherical fiducial. The registration fiducial is manually moved in the tracking space of the EMT system and the fusion Track system. The registration fiducial's 3D positions in the two coordinate frames are utilized to find a transformation matrix to map the position in the fusionTrack coordinate frame to the EMT coordinate frame.

The transformation matrix is found by using a point-to-point registration method. Three catheter tracking approaches including a pure EMT-based approach, the traditional approach (EMT+FBG) and the newly proposed approach (EMT+FBG) are used to estimate the stylus shape. The shape estimation error is calculated by the distance between each spherical fiducial's position to its closest point from the reconstructed shape.

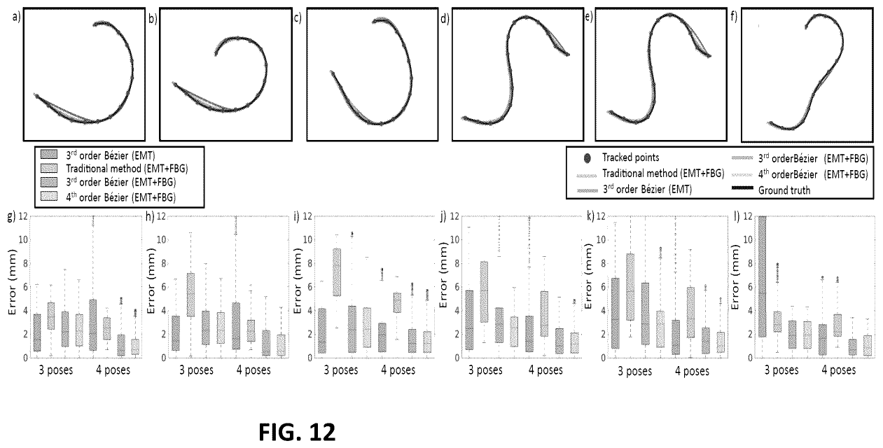

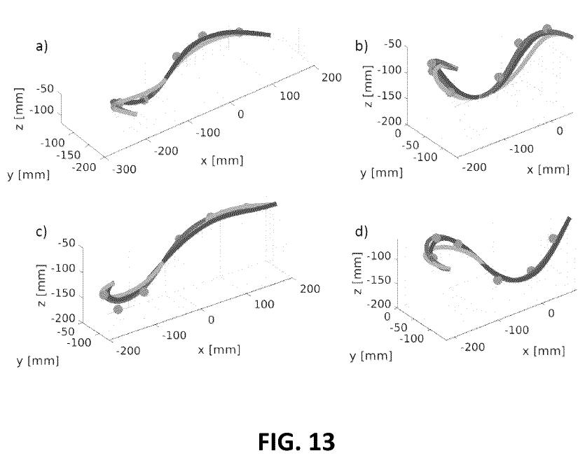

The experimental results of the 2D experiment are shown in FIG. 12. In the 1st case, 3 pose sensors are used while in the 2nd case, 4 pose sensors are used. The shapes estimated by both the traditional and the proposed method using 4 pose sensors are shown in FIG. 12(a)-(f). For each case, the shape reconstruction error of the traditional method, the proposed method using 3rd order Bézier curve and 4th order Bézier curve and the EMT-based method are presented in green, orange, cyan and magenta in FIG. 12(g)-(h), respectively. Experimental results show that the proposed method improves the tracking accuracy by approximately 50% and 70% (compared to the traditional method) in the case where 3 and 4 pose sensors are used, respectively. The largest difference in the shape tracking error between using the 3rd and 4th order Bézier curve can be seen in the 5th shape (FIG. 12(e)) where 3 pose sensors were used. This is due to the fact that an nth order Bézier curve can only change direction along an axis at most n−1 times. Hence, a 3rd order Bézier curve cannot properly represent the shape of a segment with more than two direction changes. The average shape tracking error when 3 and 4 pose sensors are used together with FBG are 2.4 mm and 0.9 mm, respectively for a 540 mm sensed length. It can be seen that the purely EMT based catheter tracking approach does not provide consistent accuracy compared to the EMT and FBG-based approaches. The EMT-based shape sensing accuracy varies depending on the complexity of the catheter shape. The 2D experimental results has shown that the 4th order Bézier curve outperforms the 3rd order Bézier curve in reconstructing a complex dilator shape. For this reason, only 4th order Bézier curve is used to estimate the 3D shape of the stylus in the 3D experiment. In the 3D experiment, the FBG stylus is bent into 4 configurations. The shape estimation results using the traditional method, the new proposed method with a 4th order Bézier curve and the EMT-based method are shown in blue, orange and yellow in FIG. 13, respectively. The mean and max shape reconstruction errors of the three methods are reported in the table below. The 3D experimental results show that by using the proposed method, the shape tracking accuracy increases by 38% compared to the traditional method.

| | EMT + FBG | | | | | |
|---|---|---|---|---|---|---|
| | | | Proposed method | | | |
| | Traditional | | (4th order | | | |
| | method | | Bezier curve) | | EMT | |
| | Mean | Max | Mean | Max | Mean | Max |
| Experiment | error | error | error | error | error | error |
| a | 8.0 ± 4.0 | 12.2 | 3.8 ± 2.9 | 7.6 | 11.9 ± 5.6 | 17.2 |
| b | 11.1 ± 3.6 | 16.6 | 5.9 ± 2.8 | 11.3 | 16.2 ± 7.0 | 26.0 |
| c | 8.4 ± 2.9 | 12.5 | 6.9 ± 3.7 | 14.1 | 10.2 ± 7.7 | 24.1 |
| d | 7.0 ± 2.2 | 10.2 | 5.1 ± 2.1 | 7.5 | 10.1 ± 6.7 | 22.6 |

The mean and max error of the reconstructed shapes in 3d experiment using the traditional method (EMT+fbg), the newly Proposed method with 4th order bézier curve (EMT+fbg) and the method in which only EMT sensors are used.

The proposed method outperforms the EMT-based catheter tracking method by 55%. Currently, the proposed algorithm is implemented in MATLAB and can run at 10 Hz and 4 Hz in the case of the 3rd and 4th order Bézier curve, respectively. Solving the optimization problem for the control points is the most time-consuming task in the proposed shape tracking algorithm. The processing time, however, can be reduced by using parallel computing while solving the optimization problems. However, one does not expect too fast shape variations in this sort of application. The most frequently used framerate of fluoroscopy for traditional minimally invasive procedures may be 7.5 Hz. The current implementation of our proposed method in case of 3^{rd} order Bézier curve is already equally fast (but does not cause radiation).

One can note that the shape tracking error of the traditional FBG-based shape sensing method reported here is significantly larger than other results reported in the art. This is due to the fact that in these works, the reconstructed shape is typically aligned with the ground truth by means of a point cloud registration (Iterative Closest Point algorithm or point-to-point registration method) before the shape sensing error is calculated. These point cloud registration methods actively reduce the error between the estimated shape and the ground truth. Unlike these previous works, the shape tracking frame (EMT coordinate frame) and the ground truth frame (fusionTrack frame) are pre-registered and we do not resort to ICP.

One of the disadvantages of the traditional FBG-based shape sensing method is that the largest shape sensing error normally appears at the tip of the fiber. This problem is caused by the fact that the shape is reconstructed by integrating the measured curvatures along the fiber length. The here proposed method does not suffer from this problem. The fiber shape is now approximated as a set of Bézier curves where the tip and the base pose of each segment are defined by EMT sensors. The proposed algorithm is general and can be applied to catheters with different numbers of integrated EMT sensors. The 2D experimental results show that by increasing the number of the integrated EMT sensors, the shape tracking accuracy tends to improve as shown in the 2D experiment (20% improvement when using 4 pose sensors compared to using 3 pose sensors).

In the present example an approach to track the 3D shape of the catheter using information provided by a multi-core optical fiber and a certain number of EMT sensors is discussed. The new approach reconstructs the catheter shape by approximating the catheter by a set of Bézier curves. Unlike the traditional FBG-based shape tracking method, where the FBG-based reconstructed shape is localized in a global frame using a point-to-point registration method, the proposed catheter shape tracking method directly reconstructs the catheter shape in the EMT coordinate frame. By approximating the catheter shape by multiple Bezier curves, the problem of the largest shape sensing error appearing at the tip of the catheter can be avoided.

Experiments in 2D and 3D have been done to verify the proposed method. The EMT-based method and the traditional EMT and FBG-based shape tracking method are also implemented to serve as a baseline. The results of the 2D experiment show that the proposed approach outperforms the traditional approach. The performance increases by approximately 50% and 70% in the case of 3 pose sensors and 4 pose sensors are used, respectively. The same pattern can be seen in the 3D experiment where the new method improves the shape tracking accuracy by approximately 38% compared to the traditional method. The combination of multi-core optical fiber and EMT sensors allows real-time tracking of the catheter during the procedure with high accuracy. The proposed approach may eliminate the need for fluoroscopy, therefore lowering harmful radiation exposure to both patients and clinicians.

### ADDITIONAL THIRD EXAMPLE

The third particular example also relates to minimally invasive catheter-based interventions which normally take place under the guidance of fluoroscopy. The present example illustrates an embodiment wherein it is not required to localize at least two EMT sensors to the multi-core fiber. Such embodiments reduce the complexity and fragility of the catheter. The embodiment illustrates a precise shape sensing approach that is robust against torsional twist. The proposed approach originates from the observation that many interventional procedures employ a plurality of concentric instruments. By distributing sensors

over these instruments, the complexity per instrument can be reduced. The proposed sensor fusion approach ensures robust and superior shape reconstruction. Experiments in 3D with ground truth generated by a stereo vision system have been done and yielded promising results, as reported here. Compared to state-of-the-art methods, embodiments of the present invention use only half of the required EMT sensors per instrument while improving the catheter shape tracking accuracy. In some examples, an improvement up to 57% was reached.

Modern surgery is increasingly relying on the Minimally Invasive Surgery (MIS). These procedures are beneficial for the patient (faster recovery, shorter hospital stay and cosmetic aspects) but they are much more challenging to perform for the physicians. In minimally invasive catheter-based interventions, long and slender instruments are inserted through a small incision and are navigated through vessels to reach the operational site. Interventions normally make use of a guidance sheath and a catheter that is coaxial with the sheath. The purpose of the guidance sheath is to offer a stable access route for the catheter toward the anatomical site of interest. At the same time, the sheath shields the fragile anatomy from excessive catheter motion. The latter can move back and forward without needing to worry that fragile regions such as aneurysms undergo excessive stresses or plaque or calcification get dislodged. Overall positioning these sheaths or navigating the catheter is hard since there is limited information on the shape and the location of the devices. Typically, fluorescence only provides 2-dimensional information. Furthermore fluorescence imaging is based on harmful radiation. It is an advantage of techniques using optical fiber sensing that fibers are small size, lightweight, have a high flexibility and provide a high degree of safety (i.e. being free from the risk of electrocution). Multi-core Fiber Bragg Grating (FBG) fibers therefore are appealing to be integrated into the flexible devices for shape sensing. Multi-core FBG fiber-based shape sensing integrates the curvatures measured at discrete points along the fiber length to reconstruct the 3D shape of the catheter. A major problem in FBG-based shape sensing is that they are not able to discriminate strain caused by twisting from strain caused by bending. Twist is regarded as one of the most significant difficulties to achieving accurate shape reconstruction since even small amounts of twist have a significant influence on the overall shape accuracy. It is an advantage of embodiments according to the present example that the number of EMT sensors that needs to be attached is reduced, e.g. minimized. It is an advantage of embodiments according to the present example that the sensors are distributed across the plurality of bodies.

In the present example, a method is illustrated to track the shapes of a plurality of flexible instruments that are arranged in a coaxial manner in the EMT coordinate frame. Embodiments according to the present example allow to track and fuse multiple instrument shapes by distributing a minimal amount of sensors across the plurality of coaxial instruments. In the present example each flexible instrument is equipped with a multi-core FBG fiber and a 5 degree-of-freedom (DOF) EMT sensor at the tip. The multi-core FBG fiber can be placed in the central or in an off-center position. The proposed method is demonstrated on a system including a 3D printed flexible guidance sheath and a catheter. The method is applicable to systems where multiple flexible instruments are organized in a coaxial fashion.

It is to be noted that features and advantages of embodiments of the present invention are not only valid for co-axial system but can be applied to systems having flexible instruments that are sufficiently parallel. If e.g. through construction the shapes measured by the pair of optical fibers can be assumed sufficiently parallel, the method according to the present example also is applicable. Evenmore, if a fixed relation between the shapes or curvature is obtained through construction, this fixed relation can also be used for correlating.

In coaxial catheter systems used in minimally invasive interventions, miniaturization is common. As a consequence, the space between the inner diameter of the guidance sheath and the outer diameter of the catheter is selected such that the inner catheter can slide back and forth inside the guidance catheter with minimal play. In such typical situation, the shapes of both bodies are-constrained to remain substantially coaxial to each other. This means that at overlapping sections, the shape of the catheter and the guidance sheath are substantially identical. In the present example, this property is utilized to co-locate the multiple sensed shapes in the same coordinate frame but also to improve the FBG-based shape sensing accuracy.

The fusion of the shapes is also shown to be advantageous to compensate for twist induced disturbances. Dynamic 3D experiments with ground truth generated by stereo vision system are done to verify the performance of the proposed coaxial catheter localization and fusion framework.

In the present example, the shape of each flexible instrument is estimated by a multi-core FBG fiber. FBG-based shape sensing relies on discrete curvature measurements made along the length of the fiber. These curvatures are obtained by employing an interrogator that measures the change in the reflected wavelength of each grating. Traditional FBG-based shape sensing methods subsequently integrate the measured curvatures to reconstruct the 3D shape of the fiber. In the present example, the catheter localization method starts with reconstructing the shape of the guidance sheath as well as the shape of the catheter in their local coordinate frames {sh} and {ca}, respectively. Such traditional FBG-based shape sensing method is briefly described below. After that, information provided by the EMT sensors attached at the tip of the guidance sheath and the catheter is incorporated to localize the two reconstructed shapes in the global EMT coordinate frame. The coaxial localization method is detailed thereafter. This approach to fuse the shapes computed from the individual fibers while taking into account the pose information offered by the two EMT sensors is explained further. The conventional FBG-based shape sensing method relies on strain measured by gratings that are distributed along the length of each optical fiber. Each grating is a Bragg reflector that reflects a particular wavelength of incoming light while transmitting the other wavelengths. The central wavelength of the reflected light of each grating is named the Bragg wavelength λ_{B}. The Bragg wavelength can be measured by an interrogator connected to the proximal end of the optical fiber. The reflected wavelength of each grating is affected by temperature ΔT and mechanical strain ε applied on the fiber. The relation between the wavelength shift Δλ, the change in surrounding temperature ΔT and the applied mechanical strain ε is given by:

$$\frac{{λ}_{B} - {λ}_{{B}_{0}}}{{λ}_{{B}_{0}}} = \frac{Δλ}{{λ}_{{B}_{0}}} = {S}_{ε} ε + {S}_{T} Δ T$$

this central core. The bending strain applied on the surrounding cores ε_{Bendi∈{2,3,4}} can then be computed as:

$${ε}_{{Bend}_{i ∈ \{ 2 , 3 , 4 \}}} = \frac{{Δλ}_{i}}{{λ}_{{B}_{0 i}} {S}_{ε}} - \frac{{Δλ}_{1}}{{λ}_{{B}_{01}} {S}_{ε}}$$

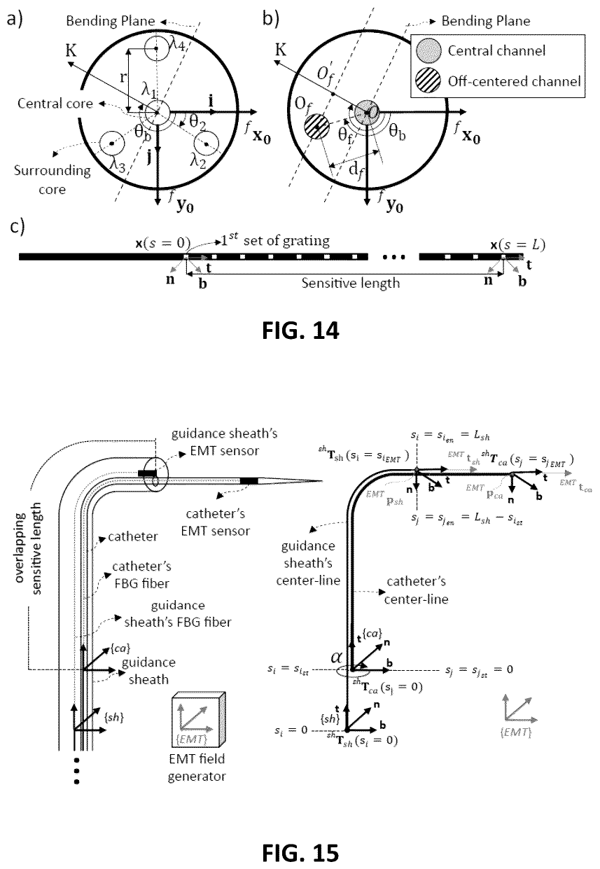

At each cross section, where a set of gratings is present, three bend-induced strains in different directions can be calculated. The relation between the curvature κ_{FBG}, the angle of bending plane θ_{bFBG} and the three calculated bend-induced strains at a given point are defined as follows:

$${ε}_{{Bend}_{i ∈ \{ 2 , 3 , 4 \}}} = - {κ}_{FBG} r sin ( {θ}_{{b}_{FBG}} - \frac{3 π}{2} - {θ}_{i} )$$

where θi is the angle of the ith core corresponding to the x-axis of the fiber; r is the distance from the surrounding core to the central core as depicted in FIG. 14A closed-form solution as known from prior art can be used to solve for the curvature κ_{FBG} and the angle of the bending plane θ_{bFBG} as below:

$${κ}_{app} = {\sum }_{i = 1}^{3} \frac{{ε}_{{Bend}_{i}}}{r} cos {θ}_{i} i - {\sum }_{i = 1}^{3} \frac{{ε}_{{Bend}_{i}}}{r} sin {θ}_{i} j ,$$

$${κ}_{FBG} = \frac{2 \vert {κ}_{app} \vert}{3} ,$$

$${θ}_{{b}_{FBG}} = {∠κ}_{app} ,$$

where i and j are the unit vectors along the x- and y-axes of the fiber's cross section (as shown in FIG. 14), respectively. In the traditional FBG-based shape sensing method, the 3D shape of the fiber is represented by a continuous and differentiable space curve. A space curve is described by curvature κ(s) and torsion τ(s) profiles, where s is the variable arc length which varies from 0 to L. The fiber sensitive length L starts at the first set of grating and ends at the last set of grating as shown in FIG. 14 part (c). Torsion τ(s) is the rate of change of the angle of the bending plane θ_{bFBG} along the fiber length s. To enhance the approximated form, the set of discrete curvatures and the angles of the bending plane are first interpolated. For each length s, a local coordinate frame with axis TNB is attached rigidly to the fiber. The evolution of the tangent t, normal n, and binormal b unit vectors (TNB frame) along the fiber arc length is determined by the estimated curvature and torsion profiles. In this work, the differential Frenet—Serret formula as known from prior art is used to solve for the evolution of the TNB frame. Finally, the position vector x(s) of each point along the fiber shape in the fiber's base frame fixed to the most proximal grating can be calculated by integrating the tangent unit vectors-as follows:

$$x ( s ) = x ( 0 ) + {\int }_{0}^{s} t ( z ) dz$$

where x(0) is the position of the fiber's base.

In the following the coaxial localization method is now focused on. The guidance sheath is sensorized by inserting a multi-core FBG fiber into its central channel. To further localize the 3D sheath shape in the EMT coordinate frame, an EMT sensor is attached to the tip of the guidance sheath. Similarly, the catheter is equipped with a multi-core fiber in the central channel and an EMT sensor at the tip. The two EMT sensors are positioned along the center-line of the guidance sheath and the catheter.

In an arbitrary situation, the guidance sheath may take on an arbitrary 3D shape, e.g. conforming to the shape of a surrounding vessel. The said shape is typically expressed by formulating the geometric shape of the center-line

$${}_{}^{sh} {x}_{sh}^{} ( {s}_{i} )$$

and the set of tangent

$${}_{}^{sh} {t}_{sh}^{} ( {s}_{i} ) ,$$

normal

$${}_{}^{sh} {n}_{sh}^{} ( {s}_{i} )$$

and binormal

$${}_{}^{sh} {b}_{sh}^{} ( {s}_{i} )$$

unit vectors with respect to the sheath's base frame {sh}. For a guidance sheath which has a sensing length of Lₛₕ, sᵢ varies from 0, at the proximal end of the sheath, to Lₛₕ, at the distal end of the sheath. The pose of each point along the centerline of the guidance sheath is denoted as

$${}_{}^{sh} {T}_{sh}^{} ( {s}_{i} ) ,$$

where

$$T ( s ) = [ \begin{matrix}t ( s ) & \tilde{n} ( s ) & b ( s ) & x ( s ) \\ 0 & 0 & 0 & 1\end{matrix} ]$$

Along similar lines, the 3D shape of the catheter can be expressed in its local reconstructed frame with respect to the base frame {ca}. The pose of each point along the center-line of the catheter is denoted as

$${}_{}^{ca} {T}_{ca}^{} ( {s}_{j} ) .$$

The arc length sⱼ of the catheter varies from 0, at the proximal end to L_{ca}, at the distal end of the sensing length of the catheter.

It is worth mentioning that in practice, the working channel of the guidance sheath through which the catheter enters is not necessarily in the center of the guidance sheath. In a more general case, the catheter thus moves in an off-centered channel. In this case, the center-line of the off-centered channel of the guidance sheath

$${}_{}^{sh} {T}_{ch}^{} ( {s}_{i} )$$

should be calculated and used in this coaxial catheter localization process. The relation of the guidance sheath center-line's curvatures and the off-centered channel center-line's curvatures is given by

$${κ}_{channel - sh} = g ( {κ}_{sh} ) = {( {{κ}_{sh}}^{- 1} - \frac{\vec{{OO}_{f}^{′}} \cdot \vec{OK}}{‖ \vec{OK} ‖} )}^{- 1} ,$$

$${θ}_{{b}_{channel - sh}} = {θ}_{{b}_{sh}} ,$$

where κₛₕ and θ_{bsh} are the curvatures and the angles of bending plane of the center-line of the guidance sheath measured by the multi-core FBG fiber, respectively. The curvatures and the angles of the bending plane of the center-line of the off-centered channel of the guidance sheath are denoted as κ_{channel-sh} and θ_{b channel-sh}, respectively.

$${O}_{f}^{′}$$

is the projection of O_{f} (center of the off-centered channel) on the bending \vec{OK} direction vector. The relative position of the off-centered channel O_{f} and the center-line of the guidance sheath O is characterized by the distance to the center-line d_{f} and the angle θ_{f} between \vec{OO_{f}} and the x-axis. The relation between these parameters can be seen in the cross sectional view of the guidance sheath shown in FIG. 14 part (B). A pre-calibration needs to be done in advance to identify the two parameters d_{f} and θ_{f}. This pre-calibration procedure is presented further below.

The pose of each point along the length of the off-centered channel center-line

$${}_{}^{ch} {T}_{ch}^{} ( {s}_{i} )$$

with respect to its base frame {ch} can be estimated by integrating the calculated κ_{channel-sh} and θ_{bchannel-sh}. A transformation matrix

$${}_{ch}^{sh} T$$

that transforms the off-centered channel shape in its base frame {ch} to the sheath base frame {sh} can then be calculated as:

$${}_{ch}^{sh} T = [ \begin{matrix} & & & - {d}_{f} cos ( {θ}_{f} ) \\ & {I}_{3 \times 3} & & - {d}_{f} sin ( {θ}_{f} ) \\ & & & 0 \\ 0 & 0 & 0 & 1\end{matrix} ]$$

To localize the reconstructed 3D shape of the catheter in the {sh} frame, a transformation matrix

$${}_{ca}^{sh} T$$

needs to be determined. Since the catheter can move backward and forward inside the off-centered tubular structure of the sheath, the origin of the catheter's base frame moves along the off-centered channel's shape in {sh}. The origin of the catheter's base frame is always aligned with a point at arc length Sist. The arc length Sist varies over time during insertion and retraction. This means that

$${}_{}^{sh} {x}_{ca}^{} ( 0 ) = {}_{}^{sh} {x}_{ch}^{} ( {s}_{{i}_{st}} ) and {}_{}^{sh} {t}_{ca}^{} ( 0 ) = {}_{}^{sh} {t}_{ch}^{} ( {s}_{{i}_{st}} ) .$$

The normal vector

$${}_{}^{sh} {n}_{ca}^{} ( 0 )$$

and binormal vector

$${}_{}^{sh} {b}_{ca}^{} ( 0 )$$

can be obtained by rotating

$${}_{}^{sh} {n}_{ch}^{} ( {s}_{{i}_{st}} ) and {}_{}^{sh} {b}_{ch}^{} ( {s}_{{i}_{st}} )$$

around

$${}_{}^{sh} {t}_{ch}^{} ( {s}_{{i}_{st}} )$$

by an angle α, respectively. Given the pose of the origin of the
catheter's base expressed in its base frame

$${}_{}^{ca} {T}_{ca}^{} ( 0 )$$

and in the sheath frame

$${}_{}^{sh} {T}_{ca}^{} ( 0 ) ,$$

a transformation matrix

$${}_{ca}^{sh} T$$

that transforms the catheter shape from its base frame {ca} to the sheath frame {sh} can be calculated by using a point-to-point registration method. By knowing shcaT, both the catheter and the guidance sheath shape can be further localized in the EMT coordinate frame {EMT}. Since the EMT manufacturer normally does not provide the actual location on the EMT sensor body that is being measured, a spatial calibration step is required to determine the correspondence of the measurement point of the EMT sensors and the FBG-based 3D reconstructed shape. By this spatial calibration step, the correspondence arc lengths of the EMT sensors attached to the guidance sheath (s_{iEMT}) and to the catheter (s_{jEMT}) can be obtained. Four points in the sheath base frame {sh}

$$a = [ \begin{matrix}{}_{}^{sh} {x}_{ca}^{} ( {s}_{{j}_{EMT}} ) & {}_{}^{sh} {x}_{ca}^{} ( {s}_{{j}_{EMT}} ) + {}_{}^{sh} {t}_{ca}^{} ( {s}_{{j}_{EMT}} ) & {}_{}^{sh} {x}_{sh}^{} ( {s}_{{i}_{EMT}} ) & {}_{}^{sh} {x}_{sh}^{} ( {s}_{{i}_{EMT}} )\end{matrix} + {}_{}^{sh} {t}_{sh}^{} ( {s}_{{i}_{EMT}} ) ]$$

together with their corresponding points in the EMT coordinate frame {EMT}

$$b = [ \begin{matrix}{}_{}^{EMT} {p}_{ca}^{} & {}_{}^{EMT} {p}_{ca}^{} + {}_{}^{EMT} {t}_{ca}^{} & {}_{}^{EMT} {p}_{sh}^{} & {}_{}^{EMT} {p}_{sh}^{}\end{matrix} + {}_{}^{EMT} {t}_{sh}^{} ]$$

can be used to perform a point-to-point registration and calculate the transformation matrix

$${}_{sh}^{EMT} T$$

that transforms the shapes reconstructed in {sh} to {EMT}. The vectors

$${}_{}^{EMT} {p}_{ca}^{} and {}_{}^{EMT} {t}_{ca}^{}$$

are the position and the unit tangent vector measured by the EMT sensor attached to the tip of the catheter.

Similarly, the position and tangent vector of the EMT sensor attached to the tip of the sheath are defined as

$${}_{}^{EMT} {p}_{sh}^{} and {}_{}^{EMT} {t}_{sh}^{} ,$$

respectively. The relation between the pose of the shape at the level (arc length s_{iEMT} and s_{jEMT}) of the EMT sensors should in principle be identical to the relation between the poses of the EMT sensors themselves. Thus, an optimization problem can be formulated to find the two unknowns sist and α to derive

$${}^{sh} {T}_{ca} ( 0 )$$

by minimizing the following cost function

$${arg min}_{{s}_{{i}_{st}} , α} ‖ {}_{sh}^{EMT} T a - b ‖$$

The two parameters sᵢₛₜ and α are varied to find optimal values that minimize the registration error of the two point clouds a and b. FIG. 15 shows the reconstructed shape of the catheter in the guidance sheath base frame {sh}.

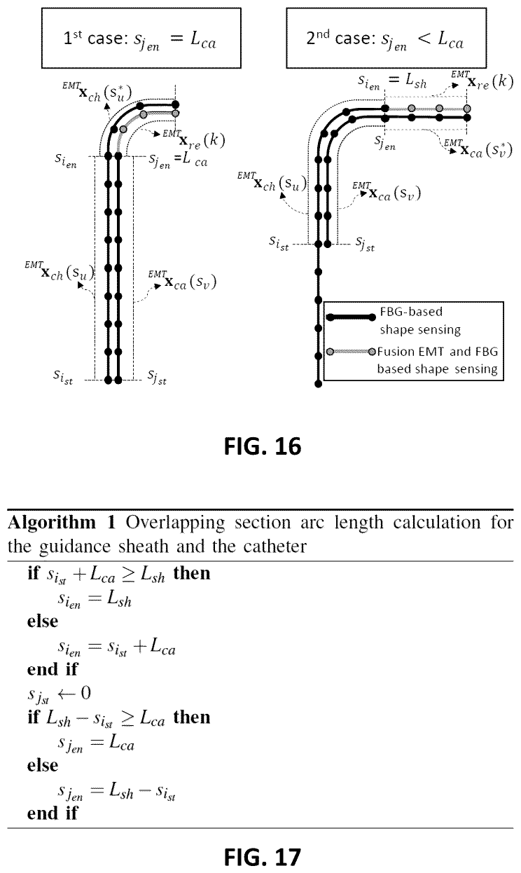

One of the disadvantages of a straight multi-core fiber compared to a helical configuration in shape sensing is that it cannot distinguish strain induced by twisting from strain induced by bending. To avoid this ambiguity, catheters are normally made from torsionally stiff elements to prevent torsion. In practice, it is very difficult to design a catheter that protects the fiber completely from torsion. Given that even small amounts of twist have a large impact on the overall shape accuracy, twist is considered one of the most important challenges to achieve precise shape reconstruction. A second disadvantage of traditional FBG-based shape sensing methods follows from the fact that the 3D shape is reconstructed by integrating the measured curvature along the fiber length. By doing so, the curvature measurement error accumulates along the length, therefore the highest shape sensing error typically appears at the fiber tip which happens to be the most interesting point one wants to know precisely. By using a coaxial catheter system (i.e. a guidance sheath and a catheter), it is possible to address these problems. Since the catheter and the sheath are coaxial, the shape of the overlapping section of the catheter and the sheath should be identical. The remaining distal section of the catheter or guidance sheath is constrained by the two EMT sensors depending on the position of the catheter with respect to the guidance sheath as can be seen in FIG. 16. In this section, a fusion framework that uses the two aforementioned characteristics to enhance the guiding sheath and catheter's shape sensing accuracy is presented. During the procedure, the catheter moves inside the guidance sheath's channel, the overlapping section can be determined by knowing the length of the two reconstructed shapes and Sist. The position of the catheter with respect to the guidance sheath's channel can belong to one of two cases. In the first case, the length of the catheter is completely covered by the guidance sheath's channel. In other words, the tip of the catheter moves inside the channel of the guidance sheath. In the second case, the tip of the catheter moves out of the guidance sheath. Assuming that the sensed length of the catheter is shorter than that of the guidance sheath (L_{ca}<Lₛₕ) and sᵢₛₜ≥0 during the procedure, the arc length of the overlapping section of the sheath (starts at sᵢₛₜ and ends at sᵢₑₙ) and of the catheter (starts at sⱼₛₜ and ends at sⱼₑₙ) as shown in FIG. 9 can be found by Algorithm 1 shown in FIG. 17.

In theory, the shape of the overlapping section in the EMT coordinate frame should be identical

$${}^{EMT} {x}_{ch} ( {s}_{u} ) = {}^{EMT} {x}_{ca} ( {s}_{v} )$$

where sᵤ={sᵢ_{ₛₜ}, . . . , sᵢ_{ₑₙ}} and sᵥ={sⱼ_{ₛₜ}, . . . , sⱼ_{ₑₙ}}. However, this is not true in practice due to the problem of curvature measurement error accumulation and dynamic twist that is applied on the fiber. A fusion framework is proposed to overcome this issue. In the here proposed fusion framework, the shape of the guidance sheath's channel and the catheter are approximated by two Bezier curves. A Bezier curve of degree n in the {EMT} frame can be specified by n+1 control points and is defined as

$${}^{EMT} b ( t ) = {\sum }_{i = 0}^{n} ( \begin{matrix}n \\ i\end{matrix} ) {( 1 - t )}^{n - i} {t}^{i} {}^{EMT} {p}_{{c}_{i}}$$

$$Where ( \begin{matrix}n \\ i\end{matrix} )$$

are the binomial coefficients and t∈[0;1]. Vectors

$${}^{EMT} {p}_{{c}_{i}}$$

are the control points.

The general idea is to find a Bezier curve that matches best with the shapes estimated by the different sensors. The shape of the overlapping section will be fused by the two shapes

$${}^{EMT} {x}_{ch} ( {s}_{u} ) and {}^{EMT} {x}_{ca} ( {s}_{v} ) .$$

The remaining section, which depending on the case can be

$${}^{EMT} {x}_{ch} ( {s}_{u}^{*} )$$

in the first case

$$( {s}_{u}^{*} = \{ {s}_{{i}_{en} + 1} , … , {L}_{sh} \} ) or {}^{EMT} {x}_{ca} ( {s}_{v}^{*} ) )$$

in the second case

$$( {s}_{v}^{*} = \{ {s}_{{j}_{en} + 1} , … , {L}_{ca} \} )$$

as shown in FIG. 16. This remaining section will be fused between the shape estimated by the traditional approach and the fusion EMT and FBG-based shape sensing approach.

Note that the shape of the remaining section can be estimated using the method since it has the two EMT sensors constraining the two ends and the discrete curvatures along the length obtained from the FBG sensors.

In the fusion EMT and FBG-based shape sensing method, the shape of the catheter segment between the two EMT sensors is represented by a Bezier curve. The first and the last control points of the Bezier curve are defined by the positions of the two EMT sensors. An optimization problem is formulated to find the remaining control points by minimizing the cost function

$${arg min}_{x = {p}_{{c}_{1}} , … , {p}_{{c}_{n - 1}}} α {E}_{length} + β {E}_{κ}$$

where α and β are the two scaling factors that regulate the relative weight of E_{length} and E_{κ}. The error E_{length} is the difference between the length of the estimated Bézier curve and the arc length between two EMT sensors. The error in curvature E_{κ} is the difference between the curvature along the length of the estimated Bézier curve and the curvatures obtained from the FBG sensors over the corresponding section. The optimization problem described in the equation given above strives to identify the control points of a Bezier curve that aligns in curvature with the curvatures measured by the FBG sensors and has the same length as the arc distance between two consecutive EMT sensors. The shape of the remaining section in the EMT coordinate frame estimated by the fusion EMT and FBG-based shape sensing method is denoted as

$${}^{EMT} {x}_{re} ( t )$$

where t∈[0;1]. The overall fused shape in the EMT coordinate frame (including the as overlapping section and the remaining section) denoted as

$${}^{EMT} {x}_{ca - fusion}$$

can now be estimated by approximating the set of points c by a Bezier curve b(t). The set of points c is defined as follows:

$$c = \{ \begin{matrix}{{}^{EMT} {x}_{ch} ( {s}_{u} ) {}^{EMT} {x}_{ch} ( {s}_{u}^{*} ) {}^{EMT} {x}_{ca} ( {s}_{v} ) {}^{EMT} {x}_{re} ( k ) ]}^{T} & ( {1}^{st} case ) \\ {{}^{EMT} {x}_{ca} ( {s}_{v} ) {}^{EMT} {x}_{ca} ( {s}_{v}^{*} ) {}^{EMT} {x}_{sh} ( {s}_{u} ) {}^{EMT} {x}_{re} ( k ) ]}^{T} & ( {2}^{nd} case )\end{matrix}$$

$$where$$

$$k = \{ \begin{matrix}\frac{{s}_{u}^{*} - {s}_{\text{?} + 1}}{{L}_{sh} - {s}_{\text{?} + 1}} & in the {1}^{st} case \\ \frac{{s}_{v}^{*} - {s}_{\text{?} + 1}}{{L}_{ca} - {s}_{\text{?} + 1}} & in the {2}^{nd} case\end{matrix}$$

$$\text{?} \text{indicates text missing or illegible when filed}$$

The order of the approximated Bezier curve EMT b(t) is defined depending on the complexity of the estimated shape given that an nth order Bezier curve can only change direction along an axis at most n−1 times. To approximate the set of m points c by a Bezier curve, the matrix form of the Bezier curve, based on the control points, can be used. The control points of 3^{rd} and 4th order Bezier curve that best fit the set of points c can be calculated.

$$[ \begin{matrix}{p}_{{c}_{0}} \\ {p}_{{c}_{2}} \\ {p}_{{c}_{2}} \\ {p}_{{c}_{3}}\end{matrix} ] = {( [ {t}^{3} {t}^{2} t 1 ] [ \begin{matrix}- 1 & 3 & - 3 & 1 \\ 3 & - 6 & 3 & 0 \\ - 3 & 3 & 0 & 0 \\ 1 & 0 & 0 & 0\end{matrix} ] )}^{+} {c}_{m \times 3}$$

$$[ \begin{matrix}{p}_{{c}_{0}} \\ {p}_{{c}_{2}} \\ {p}_{{c}_{2}} \\ {p}_{{c}_{3}} \\ {p}_{{c}_{4}}\end{matrix} ] = {( [ {t}^{4} {t}^{3} {t}^{2} t 1 ] [ \begin{matrix}1 & - 4 & 6 & - 4 & 1 \\ - 4 & 12 & - 12 & 4 & 0 \\ 6 & - 12 & 6 & 0 & 0 \\ - 4 & 4 & 0 & 0 & 0 \\ 1 & 0 & 0 & 0 & 0\end{matrix} ] )}^{+} {c}_{m \times 3}$$

where p_{c}_{_{i={0, . . . , n}}} are the control points of the Bezier curve that approximates the fused shape in the {EMT} frame. The matrix t is a m×1 matrix that contains the arc length sⱼ of each point in c normalized to the range [0;1]. It is worth noting that while the matrix forms of 3rd order and 4th order Bezier curves are given here as examples, higher order Bezier curves can still be used to approximate more complex shape depending on applications.

To validate the proposed coaxial catheter localization method and the fusion framework, 3D experiments were performed. A large diameter 3D printed steerable catheter was employed. The 3D printed catheter mimics a guidance sheath. It has a central channel that can accommodate a multi-core FBG fiber and an off-centered channel that allows a smaller catheter to be slid through. A multi-core FBG fiber with an outer diameter of 200 microns from FBGS (Geel, Belgium) was integrated into the central channel of the 3D printed guidance sheath. The fiber featured 4 cores with 8 gratings distributed along the sensitive length and present at the same arc length for each core (leading to a total of 32 gratings). The spacing between sets of gratings was 14 mm. A 3D printed fixture was made and was attached to the tip of the guidance sheath to hold the EMT sensor. An 8 Fr ablation catheter from Biosense Webster (Irvine, CA, USA) was guided by the 3Flex. This catheter was inserted into the off-centered channel of the 3D printed guidance sheath.

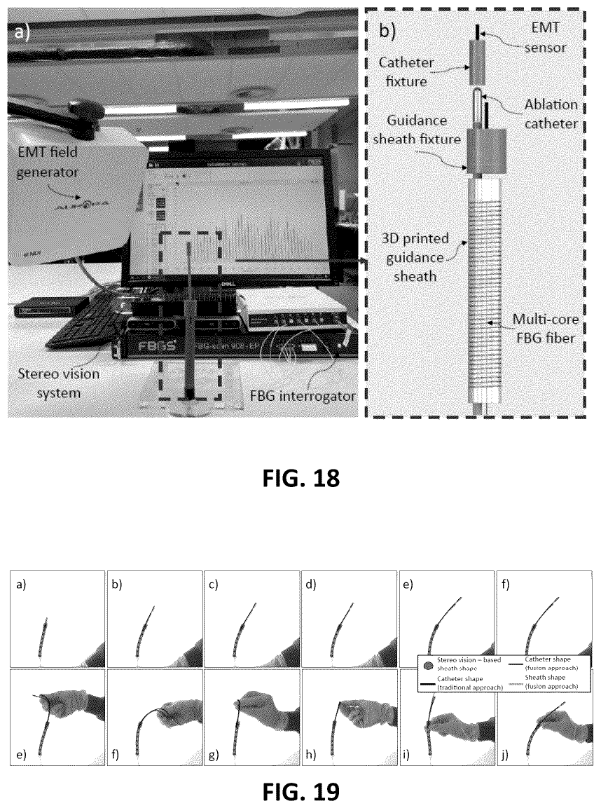

The ablation catheter fitted nicely to the guidance sheath's off-centered channel so that the catheter could simply slide forward and backward. A second 3D printed fixture was designed to fix an EMT sensor to the tip of the catheter. A multi-core FBG fiber was embedded into the irrigation channel of the ablation catheter. The used multi-core fiber had 4 cores, each with 22 gratings distributed along it. The distance between two grating sets was 10 mm. In both systems, the FBG fibers were fixed into the respective channels and glued with epoxy glue. The design of the 3D printed guidance sheath and the proposed method to integrate the fibers and the EMT sensors into the flexible instrument is shown in FIG. 18.

Two experiments have been done. In the first experiment, the catheter was inserted through the off-centered channel of the guidance sheath until the tip of the catheter reached the end point of the guidance sheath. From this point, the catheter was pushed forward and pulled backward manually. The first experiment was designed to validate the proposed coaxial catheter localization method. Thus, there was no bending applied to the catheter during the first experiment. In the second experiment, the catheter was inserted through the guidance sheath's channel until the length of the exited part of the catheter (L_{ca}−sⱼ_{ₑₙ}) was approximately 130 mm. The catheter was then manually bent into different configurations and directions. Since the purpose of this experiment is to show the effectiveness of the proposed fusion framework in improving the shape sensing accuracy, especially in large bending cases, the catheter was bent more than 90 degrees in this experiment. During the experiment, the wavelength shifts of the two multi-core fibers and the poses of the two EMT sensors were recorded to reconstruct the shape of the guidance sheath and the catheter.

Since the channel for the catheter was placed off-center, a pre-calibration step was needed to find the two parameters d_{f} and θ_{f}. By knowing these two parameters, the curvature along the center-line of the off-centered channel could be estimated from the curvature of the fiber in the central channel of the guidance sheath as described. The distance from the center line of the off-centered channel to the center line of the guidance sheath was approximately known by the design. The remaining parameter Of is the angle between \vec{OO_{f}} and the x-axis of the fiber inserted into the central channel of the guidance sheath. The pre-calibration step starts by first inserting the sensorized catheter into the off-center channel of the guidance sheath until the tip of the catheter reaches the tip of the guidance sheath. The guidance sheath is then bent in different directions manually. The wavelength shifts of both fibers are recorded during the pre-calibration. By having the catheter in the off-center channel, the curvatures of the center line of the off-center channel can be measured. To find the two parameters d_{f} and θ_{f}, an optimization problem was formulated by minimizing the following cost function

$${arg min}_{{d}_{f} , {θ}_{f}} \frac{1}{m} {\sum }_{t = 1}^{m} ‖ g ( {κ}_{{sh}_{{calib}_{\text{?}}}} ) - {κ}_{{ca}_{{calib}_{\text{?}}}} \vert$$

$$\text{?} \text{indicates text missing or illegible when filed}$$

where κ_{shcalibt} and κ_{cacalibt} are the set of curvatures measured by the fiber inserted into the sheath and by the fiber inserted into the catheter at time step tₜₕ, respectively. The number m is the number of samples recorded during the pre-calibration step (each time step is one sample).

The ground truth shapes of both the guidance sheath and the catheter were generated using two cameras (Intel RealSense D415) fixed at the same distance from the experimental setup. The experimental setup was placed in front of a white background to enhance the contrast between the catheter shape and the background. The 3D shapes of the guidance sheath were reconstructed using stereo vision system and epipolar geometry principles. The stereo vision system was calibrated using the calibration toolbox from MATLAB (The MathWorks, Inc., Massachusetts, United States). The stereo vision camera calibration process yielded a mean re-projection error of 0.28 pixels. The 3D shape of the guidance sheath could be recognized in the stereo vision coordinate frame. At each time step, the 3D guidance sheath shape was estimated by our proposed method and by the stereo vision system. To register the EMT and stereo vision coordinate frames, a point-to-point registration method was used.

Once the two coordinate frames are registered, the estimated shape of the catheter using our proposed method can be then mapped to the two image frames. The shape estimation error is calculated by the distance between each point along the mapped catheter shape to the closest point of the catheter's contour in the image frame. The guidance sheath's shape sensing error is calculated by the distance between each point of the estimated shape in the 3D stereo vision coordinate frame to the closest point of the guidance sheath ground truth shape recognized by the stereo vision. The closest points between the two sets of points were found by using the function dsearchn provided by MATLAB. Since the catheter shape is compared to its ground truth in the image frame, the unit of the shape sensing error of the catheter is in pixels while that of the guidance sheath is in mm. One may ask about the reason for having different ground truth for the guidance sheath and the catheter shape. During the experiments, the catheter experienced large bending in different directions to show the comprehensiveness of the proposed method. In some bending directions, the full shape of the catheter cannot be captured by the stereo vision system (e.g. FIG. 19 part (i) and (j)) which makes it impossible to reconstruct the full 3D catheter ground truth shape. Due to this reason, the estimated catheter shape is projected to the two image frames to calculate the shape tracking error. Unlike the catheter, the full shape of the guidance sheath is always available (since the guidance sheath did not experience large bending during the experiments), allowing for the comparison of the full guidance sheath shape in the 3D stereo vision shape reconstruction coordinate frame. The coaxial catheter localization method without the fusion framework was also implemented to be used as a baseline against which the proposed fusion framework was compared. In these experiments, only the shapes of the guidance sheath were used to register the EMT and the stereo vision shape reconstruction frame instead of using both the sheath shape and the catheter shape. Since the target of these experiments is to track the catheter's shape, this approach helps avoid the problem of actively reducing the error between the estimated catheter shape and its ground truth shape due to the point-to-point registration process.

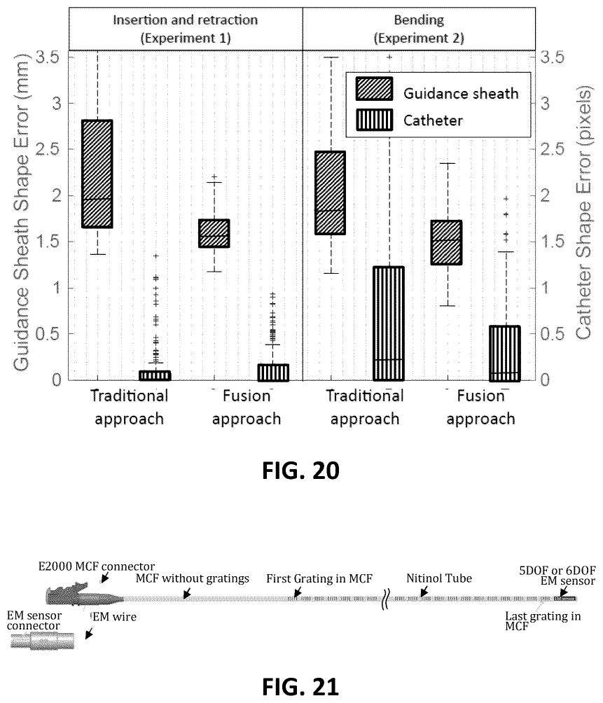

FIG. 19 shows the 3D estimated catheter shapes using the proposed fusion approach (in red) and the traditional approach (in black) projected in the left camera's images. The ground truth contours of the guidance shape and the catheter are highlighted in blue. The ground truth center lines of the guidance sheath recognized by the stereo vision system are plotted with blue markers. The green lines visualize the shapes of the guidance sheath estimated by the proposed method. The quantitative shape tracking results of the two experiments are described in FIG. 20. It can be seen that the guidance sheath mean shape sensing accuracy improves by 26% in the catheter insertion experiment (experiment 1). The same trend can be seen in the catheter bending experiment (experiment 2).

This shows the potential of the proposed coaxial catheter localization method. The mean catheter shape tracking error of the traditional approach and the fusion approach in the first experiment is similar. Unlike the first experiment, an improvement of 57% can be seen in the second experiment.

The catheter shape tracking error of both the traditional approach and fusion approach of the second experiment is larger than that of the first experiment. It is due to the bending. This may have additionally caused fiber twist, which is then potentially compensated by the new method. In the second experiment, the mean of max catheter shape sensing error reduces from 3.9 to 2.5 pixels (approximately 36%). The current framework was implemented on MATLAB and was run at 10 Hz. It is worth noting that owing to the fact that the catheter and its ground truth shapes were not aligned by using the Iterative Closest Point (ICP) approach, the reported catheter shape tracking accuracy might be larger than other works in the art which use ICP. Using ICP is avoided in this example since this registration approach will actively reduce the error between the estimated and its ground truth shapes. The two coordinate frames (EMT frame and 3D stereo vision shape reconstruction frame) were registered using the shape of the guidance shape only. The error in the shape sensing accuracy of the guidance sheath and the catheter was characterized in different units in this work. Therefore, the accuracy of the catheter and the guidance sheath tracking are not comparable. These experimental results reflect the superiority of the proposed fusion framework compared to the traditional model approach (no fusion) in combination with the coaxial catheter localization method.

In the present example, a new approach was shown to allow tracking the shapes of a plurality of flexible instruments that are arranged in a coaxial manner using a minimal number of sensors. Compared to the traditional approaches in which at least two EMT sensors are required to localize the shape of the catheter in a global coordinate frame (EMT coordinate frame), the approach herein described may co-localize multiple coaxial shapes in the same EMT coordinate frame by exploiting the coaxial property. Thanks to this property, the proposed method only requires one EMT sensor mounted to the tip of each multicore fiber. This reduces the complexity and the cost of the catheter fabrication process as well as improves the robustness of the catheter tracking system. A fusion approach is also described to improve catheter shape tracking accuracy. The presented fusion method helps compensate for the twist applied to the fiber during tracking. Experiments in 3D have been done to verify the two proposed methods with ground truth generated by the stereo vision system. The results show that the proposed coaxial catheter localization method can co-localize the guidance sheath and catheter shapes in the same coordinate frame. The described fusion approach helps improve the catheter shape tracking accuracy by 57% compared to the traditional approach in which only the coaxial catheter localization method is used.

In another example, a way to facilitate integration of a system into a catheter is discussed. The example shows that use can be made of a hybrid sensing probe consisting of a shape sensing fiber and at least one electromagnetic-sensor, e.g. at least two electromagnetic sensors. The shape sensing fiber and the at least one electromagnetic sensor, which may be one or more 5DOF and/or 6DOF EM-sensors, can be positioned inside a nitinol tube. The shape sensing fiber in the nitinol tube is fixed to a fiber optic connector but can further rotate freely inside the nitinol tube. At the end of the shape sensing fiber, the EM sensor is positioned and fixed to the nitinol tube. The nitinol tube may have an outer diameter of for example 650 μm. An example is shown in FIG. 21. The structure as discussed above can then for example be inserted inside a free lumen of a catheter.

It is to be noted that the at least one electromagnetic sensor, e.g. several electromagnetic sensors, can be positioned along the length of the shape sensing fiber, e.g. inside the nitinol tube. In a particular embodiment, the at least one electromagnetic sensors or some thereof can be positioned concentrically with the shape sensing fiber. One or more electromagnetic sensors may then surround the shape sensing fiber, for example by making use of for example a hollow electromagnetic sensor which allows to pass the fiber through the center of the electromagnetic sensor.

This illustrates how certain embodiments according to aspects of the present invention can be implemented.

1.-28. (canceled)

29. A system for determining information regarding a shape and/or pose of at least one elongated element, the system comprising:
at least one fiber optic sensor comprising a multicore optical fiber, such as for example a multicore optical fiber having multiple Fiber Bragg Gratings inscribed along the length of the multicore optical fiber,
at least two electromagnetic sensors, each of the at least two electromagnetic sensors being adapted for providing information regarding their position and/or orientation in a reference frame external to the multicore optical fiber, the at least two electromagnetic sensors being positioned with respect to the at least one fiber optic sensor so as to provide information regarding one or more distinct positions and/or orientations of the multicore optical fiber along the at least one fiber optic sensor,
a controller configured for determining information regarding the shape and/or pose of the at least one elongated element in the reference frame external to the multicore optical fiber, the controller being configured for
determining a shape and/or pose construction as function of at least one parameter, based on information from a first and/or second of the at least two electromagnetic sensors and/or based on information from the at least one fiber optic sensor, and
comparing further information of the first and/or second of the at least two electromagnetic sensors and/or of the at least one fiber optic sensor with information of the shape and/or pose construction for deriving therefrom the at least one parameter and for selecting based thereon a preferred shape and/or pose construction.

30. The system according to claim 29, wherein comparing information comprises:
varying the at least one parameter in the shape and/or pose construction, and
minimizing, as function of the parameter variation,
the difference between a curvature obtained from the shape and/or pose construction and a curvature of the at least one fiber optic sensor, and/or
a quantity depending on a position and/or orientation of one or more of the electromagnetic sensors and the shape and/or pose construction.

31. The system according to claim 29, wherein determining a shape and/or pose construction comprises determining a shape and/or pose construction as function of at least one parameter of the at least one fiber optic sensor that affects the shape and/or pose.

32. The system according to claim 31, wherein
determining a shape and/or pose construction is based on information from the first of the at least two electromagnetic sensors and information from the at least one fiber optic sensor, or
determining a shape and/or pose construction is based on information from the first of the at least two electromagnetic sensors and information from the at least one fiber optic sensor and comparing further information comprises comparing information of the second of the at least two electromagnetic sensors and information of the parameterized shape and/or pose construction for deriving therefrom the at least one parameter of the at least one fiber optic sensor that affects the shape and/or pose, or
determining comprises taking into account a predetermined relation of the at least one parameter of the fiber optic sensor that affects the shape and/or pose along the length of the at least one fiber optic sensor.

33. The system according to claim 31, wherein the controller is configured for
determining a shape and/or pose construction as function of at least one of a twist parameter and a rotation parameter of the at least one fiber optic sensor, or
determining a shape and/or pose construction as function of at least one of a twist parameter and a rotation parameter of the at least one fiber optic sensor, the predetermined relation expressing a constant twist rate along the length and/or a rotation, or
determining a shape and/or pose construction as function of at least one of a twist parameter and a rotation parameter of the at least one fiber optic sensor, the predetermined relation expressing a polynomial or other nonlinear relation of the twist rate as function of the distance along the fiber and/or a rotation.

34. The system according to claim 31, wherein determining a shape and/or pose construction comprises building up a shape and/or pose construction starting from the position of the first electromagnetic sensor and taking into account the orientation of the first electromagnetic sensor.

35. The system according to claim 31, wherein comparing information comprises varying the at least one parameter of the fiber optic sensor in a parameter space, and performing one of
minimizing a Euclidian distance between a position of the second electromagnetic sensor and a corresponding position along the shape and/or pose construction, or
minimizing a difference between an orientation of the second electromagnetic sensor and a corresponding orientation along the shape and/or pose construction, or
minimizing a difference between a position and orientation of the second electromagnetic sensor and a corresponding position respectively orientation along the shape and/or pose construction.

36. The system according to claim 33, wherein the twist parameter is a twist rate which is varied between −2π and 2π radians over the total fiber length and/or the rotation parameter is the amount of rotation which is varied between −π to π.

37. The system according to claim 29, wherein the multicore optical fiber is mounted loose end in a tube and wherein determining a shape and/or pose construction takes into account that the twist rate at the end of the multicore optical fiber is zero.

38. The system according to claim 29, wherein determining a shape and/or pose construction comprises determining the shape and/or pose construction for one, more or each section between two or more consecutive electromagnetic sensors of the at least two electromagnetic sensors as a curve, such as a Bézier curve, a B-spline, a Hermite curve, a NURBS curve, a 3rd or 4th or higher order Bézier curve, any other parameterized curve or any other implicit representation of a curve.

39. The system according to claim 38, wherein
determining a shape and/or pose construction is based at least on information from the position and orientation of the two or more consecutive electromagnetic sensors, or comparing further information comprises comparing the curvature along the parameterized curve(s) with the curvature as measured with the fiber optic sensor, or
comparing further information comprises comparing the length of the curve(s) between consecutive electromagnetic sensors with the physical length of the fiber optic sensor between the consecutive electromagnetic sensors.

40. The system according to claim 38, wherein the parameter comprises information regarding the control point(s) or any other characteristics of the curve(s).

41. The system according to claim 38, wherein the system comprises at least 3 electromagnetic sensors and wherein the shape and/or pose construction of the elongated element is built by multiple functions.

42. The system according to claim 29, wherein the first electromagnetic sensor has at least 5 DOF, and the second electromagnetic sensor has at least 3 DOF, or
wherein the at least two electromagnetic sensors are electromagnetic sensors having their longitudinal axis aligned with the longitudinal axis of one or more of the fiber optic sensors at their respective positions.

43. The system according to claim 29, wherein
the fiber optic sensor is rotationally decoupled from at least one of the at least two electromagnetic sensors, or from at least two of the at least two electromagnetic sensors, or
the fiber optic sensor is rotationally decoupled from at least one of the at least two electromagnetic sensors, or from at least two of the at least two electromagnetic sensors, the rotational decoupling of the fiber optic sensor with respect to the electromagnetic sensors being established by the fiber optic sensor being positioned loosely in a tube and the electromagnetic sensors being coupled to the tube so that they do not obstruct internal rotation of the fiber optic sensor inside the tube.

44. The system according to claim 29, wherein the position of at least one of the electromagnetic sensors can change with respect to the fiber optic sensors and wherein the position of the at least one EM sensor with respect to the fiber is dynamically updated in the controller.

45. The system according to claim 29, wherein one electromagnetic sensor is positioned with respect to one fiber optic sensor and wherein the second electromagnetic sensor is positioned with respect to another fiber optic sensor, and wherein the controller is adapted for correlating the position of the two electromagnetic sensors with respect to the at least one of the fiber optic sensors.

46. The system according to claim 29, wherein the controller is adapted for correlating the position of the two electromagnetic sensors with respect to the at least one of the fiber optic sensors based on shape and/or pose information obtained from a coinciding portion of both fiber optic sensors or from historic data, or from knowledge of the relative motion of the fiber optic sensors and/or electromagnetic sensors e.g. by some external measurement system.

47. The system according to claim 29, wherein at least one of the at least two electromagnetic sensors may be a virtual electromagnetic sensor representing a historic or memorized position and/or pose of a non-virtual electromagnetic sensor.

48. A method for determining information regarding a shape and/or pose of at least one elongated element, the method comprising:
determining a shape and/or pose construction as function of at least one parameter, based on information from a first and/or second of at least two electromagnetic sensors and/or based on information from at least one fiber optic sensor, and
comparing further information of the first and/or second of the electromagnetic sensors and/or of the at least one fiber optic sensor with information of the shape and/or pose construction for deriving therefrom the at least one parameter and for selecting based thereon a preferred shape and/or pose construction.
